(19) United States
(12) Patent Application Publication
(10) Pub. No.: US 2023/0364227 A1
(43) Pub. Date: Nov. 16, 2023

(54) POXVIRUS ADJUVANT FOR T-CELL VACCINATION

(71) Applicant: Pellis Therapeutics, Inc., Boston, MA (US)

(72) Inventors: Thomas S. Kupper, Boston, MA (US); Eric Stromquist, Boston, MA (US)

(21) Appl. No.: 18/315,906

(22) Filed: May 11, 2023

Related U.S. Application Data
(60) Provisional application No. 63/341,036, filed on May 12, 2022.

(51) Int. Cl. A61K 39/39; A61K 39/285; A61K 39/00

(52) U.S. Cl. CPC A61K 39/39; A61K 39/285; A61K 39/4611; A61K 2039/54; A61K 2039/572

(57) ABSTRACT

Intact, non-replicating or replication-deficient poxvirus acts as an adjuvant when administered with mechanical disruption, with or without a T cell antigen. Compositions and methods for inducing T cell mediated immune responses to antigen in epithelial tissues of a subject are provided.

### CROSS-REFERENCE TO RELATED APPLICATIONS

This application claims the benefit of and priority to U.S. Provisional Application No. 63/341,036 filed May 12, 2022, which is hereby incorporated by reference in its entirety.

### STATEMENT REGARDING FEDERALLY SPONSORED RESEARCH OR DEVELOPMENT

None.

### FIELD OF THE INVENTION

The invention is generally in the area of using a non-replicating or replication-impaired virus as an immune adjuvant, optionally in combination with a T cell antigen.

### BACKGROUND OF THE INVENTION

Vaccination approaches against pathogens such as HIV, HCV, TB, malaria and dengue fever, as well as cancer, may be enhanced by inducing T cell-mediated immunity (TCMI). Inactivated viral vaccines have generally proven ineffective in producing TCMI, suggesting the need for viable active viral vectors. However, although vaccination approaches employing intact active virus as a vector may induce TCMI, these viruses have been associated with the development of diseases and disorders amongst vaccine recipients, especially in immunocompromised subjects. In addition, the development of intact viable viruses as vaccines expressing endogenous antigen is often time-consuming and labor-intensive, rendering such approaches impractical for combatting epidemic or pandemic diseases.

Increasing a T cell mediated immune response would be advantageous .

It is therefore an object of the present invention to provide reagents and methods for stimulating a T cell mediated immune response.

It is a further object of the invention to provide more effective vaccines that can be readily prepared and administered to provide a protective T cell immune response against a wide range of pathogens and cancers.

### SUMMARY OF THE INVENTION

It has been established that co-delivery of intact non-replicating or replication-deficient poxvirus in combination with a T cell antigen gives rise to antigen-specific T cell mediated immunity (TCMI) to the antigen. The T cell antigen may be co-administered, or be present in, the disrupted epithelial tissue in a subject into which the replication-deficient poxvirus is administered.

Methods for inducing or stimulating a T cell mediated immune response to a T cell antigen in epithelial tissues of a human subject include administering to disrupted epithelial tissue of the subject an intact, non-replicating or replication-impaired poxvirus, in combination with a T cell antigen, or a vector expressing the T cell antigen. Typically, the intact, non-replicating or replication-impaired poxvirus and the T cell antigen, or vector expressing the T cell antigen, are administered at the same time that the epithelial tissue is disrupted. In preferred embodiments, the intact, non-replicating or replication-impaired poxvirus is derived by natural or artificial modification of a wild-type poxvirus. Exemplary poxviruses include orthopox, suipox, avipox, capripox, leporipox, parapoxvirus, molluscpoxvirus, and yatapoxvirus. A preferred orthopox virus to be modified is a vaccinia virus. Exemplary vaccinia viruses include Modified Vaccinia Ankara (MVA), Wyeth strain, WR strain, NYCBH strain, ACAM2000, Lister strain, LC16m8, Elstree-BNm, Copenhagen strain, and Tiantan strain. A preferred vaccinia virus is Modified Vaccinia Ankara (MVA).

In some embodiments the methods include co-administering the poxvirus with a T cell antigen that is a protein, a polypeptide, or a nucleic acid encoding for the above. In a preferred embodiment the T cell antigen is a polypeptide, or a nucleic acid encoding a polypeptide. An exemplary polypeptide is a HLA-restricted peptide, such as an epitope for a MHC class I peptide. In other embodiments, the T cell antigen is administered in the form of a vector separate from the poxvirus that expresses the T cell antigen within the recipient. Exemplary vectors include a nucleic acid plasmid, a nucleic acid cosmid, replicon RNA, a viral vector, a virus-like-particle (VLP), liposomal nucleic acid, an inactivated prokaryotic cell, an inactivated fungal cell, a eukaryotic cell, and an artificial chromosome. Exemplary viral vectors include lentivirus, retrovirus, adenovirus, Adeno-associated virus (AAV) and alphavirus. In another embodiment, the T-cell antigen is present in or adjacent the site of administration of the poxvirus, for example, when administered intratumorally or near to the tumor or a site where the tumor has been excised or treated with a cytolytic agent.

In some embodiments, the subject has or is at risk of developing cancer, and the T cell antigen is a tumor-associated antigen (TAA), a tumor-specific antigen (TSA), or a tissue-specific antigen. In some embodiments, the T cell antigen is a tumor neoantigen or a MHC neo-epitope. In some embodiments, the T cell antigen is a tumor antigen derived from epithelial tissue, such as the skin, oral mucosa, esophagus, reproductive mucosa and urogenital mucosa. In other embodiments, the subject has or is at risk of developing a viral, bacterial, fungal, or protozoal infection, and the T cell antigen is a viral, bacterial, fungal or protozoal antigen. A preferred pathogen-derived T cell antigen raises a protective immune response against a coronavirus, such as the SARS-Cov-2 virus.

The vaccine is administered into epithelial tissue, for example, skin, lung, oral mucosa, gastrointestinal tract, rectal or vaginal mucosa. A preferred epithelial tissue is skin tissue. Typically, epithelial skin tissue is disrupted to the top layer of dermis, leading to pinpoint bleeding, and non-replicating or replication-impaired poxvirus and T cell antigen, or vector expressing the T cell antigen are administered to the epidermis abutting the dermis.

In some embodiments, the epithelial tissue is mechanically disrupted by a scarification needle, a needle, an abrader or microneedles. In some embodiments, the epithelial tissue is disrupted essentially at the same time as the administration of the poxvirus and the T cell antigen, or the vector expressing the T cell antigen. In other embodiments, the epithelial tissue is disrupted before administration of the poxvirus and the T cell antigen, or the vector expressing the T cell antigen. In some embodiments, the methods deliver the poxvirus to a first disrupted epithelial tissue location on the subject, and deliver the T cell antigen, or vector expressing the T cell antigen, to a second disrupted epithelial tissue location of the subject. In some embodiments, the type of epithelial tissue of the first disrupted epithelial tissue location is different from that of the second disrupted epithelial tissue location.

In some embodiments, the methods include administering to the subject, or co-expressing within the subject, a co-stimulatory molecule, a growth factor, or a cytokine. The methods include administering the molecule to the subject before, at the same time, and/or after the poxvirus and T cell antigen is administered.

Kits of reagents for providing a T cell-mediated immune response to an antigen in a subject are also provided. The kits may include two or more of a device for mechanically disrupting a subject’s epidermal tissue; an intact, non-replicating or replication-impaired poxvirus delivering a T cell antigen, and T cell antigen, in an amount sufficient to stimulate an immune response when administered to disrupted epidermal tissue. Examples of antigen formulation include nucleic acids expressing the antigen, in a carrier such as a lipid carrier.

Dosage units of reagents for providing a T cell-mediated immune response to an antigen in a subject by mechanical disruption of an epidermal tissue are also provided. In some embodiments, the dosage units include an effective amount of antigen for inducing or stimulating a protective T cell mediated immune response to a T cell antigen in epithelial tissues such as skin, lung, oral mucosa, gastrointestinal tract, and reproductive mucosa comprising an intact, non-replicating or replication-impaired poxvirus and a T cell antigen, or vector expressing the T cell antigen.

### DETAILED DESCRIPTION OF THE INVENTION

### I. Definitions

The terms “immunologic”, “immunological” or “immune” response refer to the development of a beneficial cellular (mediated by antigen-specific T cells and their secretion products) response directed against an immunogen in a recipient patient. Such a response can be an active response induced by administration of immunogen or a passive response induced by administration primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

The term “T cell antigen” refers to a protein or fragment thereof which can be processed into a peptide that can bind to either Class I MHC, Class II MHC, non-classical MHC, or CD1 family molecules (collectively antigen presenting molecules), and in this combination can engage a T cell receptor on a T cell. Accordingly, a T cell mediated immune response is a response that occurs as a result of recognition by the T cell receptor of a T cell antigen bound to an antigen presenting molecule on the cell surface of an antigen presenting cell, coupled with other interactions between costimulatory molecules on the T cell and APC. This response serves to induce T cell proliferation, anatomic migration, and production of effector molecules, including cytokines and other factors that can injure cells.

The term “B cell antigen” refers to a protein, glycoprotein, carbohydrate, or lipid that can bind to cell surface antibody and can generate the production of soluble antibodies. A humoral immune response is the generation of an immune response that leads to high and sustained levels of circulating antibodies.

The term “treat” or “treatment” of a disease, disorder or condition refers to improving one or more symptoms or the general condition of a subject having the disease. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating or palliating the disease state, and remission or improved prognosis. In the case of cancer, “treating” the cancer refers to inhibiting proliferation or metastasis of a cancer or tumor cells. In some embodiments, treatment leads to stasis, partial or complete remission of a tumor or inhibit metastatic spreading of the tumor. In the case of an infectious disease, “treating” the infectious disease means reducing the load of the infectious agent in the subject. In some embodiments, the load is viral load, and reducing the viral load means, for example, reducing the number of cells infected with the virus, reducing the rate of replication of the virus, reducing the number of new virions produced or reducing the number of total viral genome copies in a cell, as compared to an untreated subject. ?

The terms “effective amount” or “therapeutically effective amount” mean a dosage or other amount of an active agent that produces some desired effect at a reasonable benefit/risk ratio applicable to any medical treatment or diagnosis. Typically, an amount of an agent is therapeutically effective if it is sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered. One of ordinary skill in the art may empirically determine the effective amount of a particular compound without necessitating undue experimentation.

The terms “pharmaceutically acceptable” or “biocompatible” refer to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. The term “pharmaceutically acceptable salt” is art-recognized, and includes relatively non-toxic, inorganic and organic acid addition salts of compounds

The terms “inhibit” or “reduce” generally mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%, or an integer there between. In some embodiments, the inhibition and reduction are compared at mRNAs, proteins, cells, tissues and organs levels.

The terms “prevent”, “prevention” or “preventing” mean to administer a composition or method to a subject or a system at risk for or having a predisposition for one or more symptom caused by a disease or disorder, to decrease the likelihood the subject will develop one or more symptoms of the disease or disorder, or to reduce the severity, duration, or time of onset of one or more symptoms of the disease or disorder.

The terms “protein” “polypeptide” or “peptide” refer to a natural or synthetic molecule comprising two or more amino acids linked by the carboxyl group of one amino acid to the alpha amino group of another.

The term “polynucleotide” or “nucleic acid” or “nucleic acid sequence” refers to a natural or synthetic molecule comprising two or more nucleotides linked by a phosphate group at the 3′ position of one nucleotide to the 5′ end of another nucleotide. The polynucleotide is not limited by length, and thus the polynucleotide can include deoxyribonucleic acid (DNA) or ribonucleic acid (RNA).

Use of the term “about” is intended to describe values either above or below the stated value in a range of approx. +/- 10%; in other embodiments the values may range in value either above or below the stated value in a range of approx. +/- 5%.

### II. Compositions for T-Cell Vaccination via Epidermal Disruption

Administration of intact non-replicating or replication-deficient poxvirus, in combination with a T cell antigen at or near the site of administration of the poxvirus, to disrupted epithelial tissue induces a T cell mediated immune response specific to the T cell antigen in a subject. Typically, compositions for inducing antigen-specific T cell mediated immunity include intact, non-replicating or replication-impaired poxvirus and one or more T cell antigens or molecules expressing one or more T cell antigens. Exemplary T cell antigens include viral or tumor antigens. In some embodiments, one or more additional molecules enhances or induces an immune response when combined with intact, non-replicating or replication-impaired poxvirus and one or more T cell antigens. Exemplary additional molecules include co-stimulatory molecules, growth factors, and cytokines.

### A. Non-replicating or Replication-impaired Poxvirus

Several different replication deficient or modified, non-replicating or replication-impaired poxvirus can be used. The viruses have a low replicative efficiency in the target cell, which prevents sustained replication and infection of other cells.

Poxviruses are useful as vaccines to generate immune responses, for the development of new vaccines, for delivery of desired proteins and for gene therapy. The advantages of poxvirus vectors include: (i) ease of generation and production, (ii) the stability of the virus, (iii) efficient infection and presentation of viral antigen in multiple cell types, including antigen-presenting cells, (iv) high levels of protein expression, (v) optimal presentation of antigens to the immune system, and (vi) the ability to elicit cell-mediated immune responses as well as antibody responses, (vii) the ability to use combinations of poxviruses from different genera, as they are not immunologically cross-reactive and (viii) the long-term experience gained with using this vector in humans as a smallpox vaccine. Poxviruses are well known cytoplasmic viruses. As a result of the non-integrative cytoplasmic nature of the poxvirus, the poxvirus will not result in having long-term persistence in other cells.

In preferred embodiments, the intact non-replicating or replication-impaired poxvirus is derived by natural or artificial modification of a wild-type poxvirus. Exemplary poxviruses include orthopox, suipox, avipox, capripox, leporipox, parapoxvirus, molluscpoxvirus, and yatapoxvirus. A preferred orthopox virus is a vaccinia virus. Exemplary vaccinia viruses include Modified Vaccinia Ankara (MVA), Wyeth strain, WR strain, NYCBH strain, ACAM2000, Lister strain, LC16m8, Elstree-BNm, Copenhagen strain, and Tiantan strain. A preferred vaccinia virus is Modified Vaccinia Ankara (MVA).

The term “modified” poxvirus refers to a poxvirus that has been altered in some way that changes one or more characteristics of the modified virus compared to the wild-type virus. These changes may have occurred naturally or through engineering. In some embodiments, the modified virus is altered to include one or more cytokines, co-stimulatory molecules, or adjuvants. In preferred embodiments, the poxvirus is not modified.

The term “non-replicating” or “replication-impaired” poxvirus refers to a poxvirus that is not capable of replication to any significant extent in the majority of normal mammalian cells or normal primary human cells. The term “significant extent” means a replication capability of 75% or less as compared to wild-type vaccinia virus in standardized assays. In some embodiments, the poxvirus has a replication capability of 65%, 55%, 45%, 35%, 25%, or 15% compared to wild-type vaccinia virus. In some embodiments, the virus has a replication capability 10% or less, 5% or less, or 1% or less compared to wild-type virus. Non-replicating viruses are 100% replication deficient in normal primary human cells. The replication deficient, or non-replicating, or replication-impaired poxviruses are intact and viable particles, as opposed to virus that has been physically or chemically-inactivated, for example, by exposure to formalin or β-propiolactone, to destroy infectivity.

Viral replication assays are known in the art, and can be performed for vaccinia viruses on e.g. primary keratinocytes, and are described in Liu et al. J.Virol. 2005, 79:12, 7363-70. Viruses which are non-replicating or replication-impaired may have become so naturally (i.e. they may be isolated as such from nature) or artificially e.g. by breeding in vitro or by genetic manipulation, for example deletion of a gene which is critical for replication. There will generally be one or a few cell types in which the viruses can be grown, such as CEF cells for MVA.

In some embodiments, the modified poxvirus may also have altered characteristics concerning aspects of the viral life cycle, such as target cell specificity, route of infection, rate of infection, rate of replication, rate of virion assembly and/or rate of viral spreading.

### 1. Vaccinia Virus

In some embodiments, the modified poxvirus is a Vaccinia virus (VV). Vaccinia virus (VV) is the prototype of the genus Orthopoxvirus. It is a doublestranded DNA (deoxyribonucleic acid) virus that has a broad host range under experimental conditions (Fenner et al. Orthopoxviruses. San Diego, Calif.: Academic Press, Inc., 1989; Damaso et al., Virology 277:439-49 (2000)).

A number of poxviruses have been developed as live attenuated vaccinia virus strains, including Modified Vaccinia Ankara (MVA) and Wyeth (Cepko et al., Cell 37:1053 1062 (1984); Morin et al., Proc. Natl. Acad. Sci. USA 84:4626 4630 (1987); Lowe et al., Proc. Natl. Acad. Sci. USA, 84:3896 3900 (1987); Panicali & Paoletti, Proc. Natl. Acad. Sci. USA, 79:4927 4931(1982); Mackett et al., Proc. Natl. Acad. Sci. USA, 79:7415 7419 (1982)). Other attenuated vaccinia virus strains include WR strain, NYCBH strain, ACAM2000, Lister strain, LC16m8, Elstree-BNm, Copenhagen strain, and Tiantan strain.

### A. Modified Vaccinia Virus Ankara (MVA)

In some embodiments, the modified poxvirus is a Modified vaccinia virus Ankara (MVA), or a derivative thereof. Modified vaccinia virus Ankara, and derivative strains have been generated by long-term serial passages of the Ankara strain of vaccinia virus (CVA) on chicken embryo fibroblasts (for review see Mayr, A., et al., Infection, 3:6-14 (1975). The MVA virus may be obtained from public repository sources. For example, MVA was deposited in compliance with the requirements of the Budapest Treaty at CNCM (Institut Pasteur, Collection Nationale de Cultures Microorganisms, 25, rue du Docteur Roux, 75724 Paris Cedex 15) on Dec. 15, 1987 under Depositary No. I-721 (U.S. Pat. No. 5,185,146); MVA virus was deposited in compliance with the Budapest Treaty at the European Collection of Cell Cultures (ECACC) (CAMR, Porton Down, Salisbury, SP4 OJG, UK) on Jan. 27, 1994, under Depository No. V94012707) (U.S. Pat. No. 6,440,422 and U.S. Pat. Publication No. 2003/0013190). Also, U.S. Pat. Publication No. 2003/0013190 further discloses particular MVA strains deposited at the ECACC under Depository No. 99101431, and ECACC provisional accession number 01021411. Commercially available are THERION-MVA, THERION PRIFREE vectors and THERION M-SERIES vectors (Therion Biologics Corporation, MA).

MVA was generated by 516 serial passages on chicken embryo fibroblasts of the Ankara strain of vaccinia virus (CVA) (Mayr, A., et al. Infection 3, 6-14 [1975]). As a consequence of these long-term passages, about 31 kilobases of the genomic sequence were deleted from the virus (deletion I, II, III, IV, V, and VI) and, therefore, the resulting MVA virus was described as being highly host cell restricted to avian cells (Meyer, H. et al., J. Gen. Virol. 72, 1031-1038 [1991]). It was shown in a variety of animal models that the resulting MVA was significantly avirulent (Mayr, A. & Danner, K. [1978] Dev. Biol. Stand. 41: 225-34). Additionally, this MVA strain has been tested in clinical trials as a vaccine to immunize against the human smallpox disease (Mayr et al., Zbl. Bakt. Hyg. I, Abt. Org. B 167, 375-390 [1987], Stickl et al., Dtsch. med. Wschr. 99, 2386-2392 [1974]). These studies involved over 120,000 humans, including high-risk patients, and proved that compared to vaccinia based vaccines, MVA had diminished virulence or infectiousness while it induced a good specific immune response. Generally, a virus strain is regarded as attenuated if it has lost its capacity or only has reduced capacity to reproductively replicate in host cells.

Because the genome of both wild type VV and MVA have both been sequenced, it is possible to clone viruses that bear some resemblance to MVA with regard to replication properties, but are genetically distinct from MVA. These may serve the same purpose, or may be more immunogenic than MVA while being just as safe by virtue of their replication deficiency.

In some embodiments, changes in the virus include, for example, alterations in the gene expression profile of the virus. In some embodiments, the modified virus may express genes or portions of genes that encode peptides or polypeptides that are foreign to the poxvirus, i.e., would not be found in a wild-type virus. These foreign, heterologous or exogenous peptides or polypeptides can include sequences that are immunogenic such as, for example, tumor-specific antigens (TSAs), bacterial, viral, fungal, and protozoal antigens, or antigenic sequences derived from viruses other than the viral vector. The genetic material may be inserted at an appropriate site within the virus genome for the recombinant virus to remain viable, i.e. the genetic material may be inserted at a site in the viral DNA (e.g., non-essential site in the viral DNA) to ensure that the recombinant virus retains the ability to infect foreign cells and to express DNA, while maintaining the desired immunogenicity and diminished virulence. For example, as described above, MVA contains 6 natural deletion sites which have been demonstrated to serve as insertion sites. See, for example, U.S. Pat. No. 5,185,146, and U.S. Pat. No. 6,440,422. In some embodiments, genes that code for desired antigens are inserted into the genome of a poxvirus in such a manner as to allow them to be expressed by that virus along with the expression of the normal complement of parent virus proteins.

### B. T Cell Antigens

Antigens are compounds or derivatives thereof that are specifically bound T lymphocyte antigen receptors.

AT cell immunogen is an antigen (or adduct) that is able to trigger a cell-mediated immune response. It first initiates an innate immune response, which then causes the activation of the adaptive immune response. An antigen binds the highly variable immunoreceptor products (B cell receptor or T cell receptor) once these have been generated. Immunogens are those antigens, termed immunogenic, capable of inducing an immune response. However, unless specifically indicated otherwise, any of the antigens can also be an immunogenic (i.e., an immunogen). As used herein, “antigen” includes the actual antigen or a nucleic acid molecule encoding the antigen.

Antigens are selected or designed for immune stimulation or immune tolerance, of B-cells and/or T-cells, with or without the context of an MHC complex. In some embodiments, the antigens are those suitable for MHC complex presentation by APC such as dendritic cells at or around the site of administration.

Antigens can be or can include, for example, peptides or proteins, nucleic acids,. Exemplary antigens include B cell antigens and T cell antigens. B cell antigens can be peptides, proteins, polysaccharides, saccharides, lipids, nucleic acids, small molecules (alone or with a hapten) or combinations thereof. T cell antigens are typically proteins or peptides. In a preferred embodiment, the T cell antigen is a polypeptide or a nucleic acid encoding a polypeptide (mRNA). The antigen can be derived from a virus, bacterium, parasite, plant, protozoan, fungus, tissue or transformed cell such as a cancer or leukemic cell or immunogenic component thereof, e.g., cell wall components or molecular components thereof. The antigens can be allergens, environmental antigens or tumor antigens. The antigen can be associated with one or more diseases or conditions such as infectious diseases, autoimmune diseases, and cancer.

Suitable antigens are known in the art and are available from commercial government and scientific sources. The antigens can be purified or partially purified polypeptides derived from tumors or viral or bacterial sources. The antigens can be recombinant polypeptides produced by expressing DNA or mRNA encoding the polypeptide antigen in a heterologous expression system. Antigens can be provided as single antigens or can be provided in combination. Antigens can also be provided as complex mixtures of polypeptides or nucleic acids.

In some embodiments, the antigen is a viral antigen or nucleic acid encoding a viral antigen. A viral antigen can be isolated from any virus. In an exemplary embodiment, the antigen is a natural viral capsid structure. In some embodiments, the antigen is a bacterial antigen. Bacterial antigens can originate from any bacteria. In some embodiments the antigen is a parasite antigen. In some embodiments, the antigen is an allergen or environmental antigen. Exemplary allergens and environmental antigens, include, but are not limited to, antigens derived from naturally occurring allergens such as pollen allergens (tree-, herb, weed-, and grass pollen allergens), insect allergens (inhalant, saliva and venom allergens), animal hair and dandruff allergens, and food allergens. In some embodiments, the antigen is a self-antigen such as in immune tolerance applications for auto-immune or related disorders such as Multiple Sclerosis. In some embodiments, the antigen is a tumor antigen or nucleic acid encoding a tumor antigen. Exemplary tumor antigens include a tumor-associated or tumor-specific antigen.

T cells respond to threats in an antigen-specific manner using T cell receptors (TCRs) that recognize short peptide antigens presented on major histocompatibility complex (MHC) proteins. The TCR-peptide-MHC interaction mediated between a T cell and its target cell dictates its function and thereby influences its role in disease. In preferred embodiments, the antigen is a T cell antigen. In some embodiments, the T cell antigen is one that requires processing such as proteolytic cleavage by antigen-presenting cell before it can be recognized by the T lymphocytes.

In further embodiments, the antigens are any approved vaccines that are designed to elicit an immune response to protect again a particular pathogen. Vaccines include, but not limited to, whole-pathogen vaccines such as inactivated viruses, live-attenuated viruses, and chimeric vaccine; subunit vaccines such as protein subunit vaccines, peptide vaccines, virus-like particles (VLPs), and recombinant proteins; and nucleic acid-based vaccines such as DNA plasmid vaccines, mRNA vaccines, and recombinant vector vaccines utilizing viral expression vectors. Exemplary vaccines include Adenovirus Type 4 and Type 7 Vaccine, ERVEBO® (Ebola Zaire Vaccine, Live), DENGVAXIA® (Dengue Tetravalent Vaccine, Live), DAPTACEL® (Diphtheria and Tetanus Toxoids and Acellular Pertussis Vaccine), M-M-R II® (Measles, Mumps, and Rubella Virus Vaccine Live), TRUMENBA® (Meningococcal Group B Vaccine), POLIOVAX® (Poliovirus Vaccine Inactivated), IMOVAX® (Rabies Vaccine), RABAVERT® (Rabies Vaccine), ROTARIX® (Rotavirus Vaccine, Live), JYNNEOS® (Smallpox and Monkeypox Vaccine, Live), TYPHIM Vi® (Typhoid Vi Polysaccharide Vaccine), and YF-VAX® (Yellow Fever Vaccine). Exemplary COVID-19 vaccines include Pfizer-BioNTech COVID-19 vaccine, Moderna COVID-19 vaccine, Oxford/AstraZeneca COVID-19 vaccine, Russia’s Sputnik V COVID-19 vaccine, and Chinese Sinopharm COVID-19 vaccine.

### 1. Viral Antigens

In some embodiments, the antigen is a viral antigen isolated from a virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenza virus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens can be derived from a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

Exemplary viral antigens include influenza virus hemagglutinin (Genbank accession No. JO2132; Air, 1981, Proc. Natl. Acad. Sci. USA 78:7639-7643; Newton et al., 1983, Virology 128:495-501), influenza virus neuraminidase, PB1, PB2, PA, NP, M₁, M₂, NS₁, NS₂)) of Influenza A or B virus; E1A, E1B, E2, E3, E4, E5, L1, L2, L3, L4, L5 of Adenovirus; Pneumonoviridae (e.g., pneumovirus, human respiratory syncytial virus): Papovaviridae (polyomavirus and papillomavirus): E1, E2, E3, E4, E5a, E5b, E6, E7, E8, L1, L2; Human respiratory syncytial virus: human respiratory syncytial virus: G glycoprotein (Genbank accession no. Z33429; Garcia et al., 1994, J. Virol.; Collins et al., 1984, Proc. Natl. Acad. Sci. USA 81:7683), RSV-viral proteins, e.g., RSV F glycoprotein; Dengue virus: core protein, matrix protein or other protein of Dengue virus (Genbank accession no. M19197; Hahn et al., 1988, Virology 162:167-180); Measles: measles virus hemagglutinin (Genbank accession no. M81899; Rota et al., 1992, Virology 188:135-142); Herpesviridae (e.g., herpes simplex virus 1, herpes simplex virus 2, herpes simplex virus 5, and herpes simplex virus 6: herpes simplex virus type 2 glycoprotein gB (Genbank accession no. M14923; Bzik et al., 1986, Virology 155:322-333), gB, gC, gD, and gE, HIV (GP-120, p17, GP-160, gag, po1, qp41, gp120, vif, tat, rev, nef, vpr, vpu, vpx antigens), ribonucleotide reductase, α-TIF, ICP4, ICP8, 1CP35, LAT-related proteins, gB, gC, gD, gE, gH, gI, gJ, and dD antigens; Lentivirus (e.g., human immunodeficiency virus 1 and human immunodeficiency virus 2): envelope glycoproteins of HIV I (Putney et al., 1986, Science 234:1392-1395)

Picornaviridae (e.g., enterovirus, rhinovirus, hepatovirus (e.g., human hepatitis A virus); Cardiovirus; Apthovirus; Reoviridae (orthoreovirus, orbivirus, rotavirus, cypovirus, fijivirus, phytoreovirus, and oryzavirus), Retroviridae (mammalian type B retroviruses, mammalian type C retroviruses, avian type C retroviruses, type D retrovirus group, BLV-HTLV retroviruses); spumavirus, flaviviridae (e.g., hepatitis C virus), hepadnaviridae (e.g., hepatitis B virus), togaviridae (e.g., alphavirus (e.g., sindbis virus) and rubivirus (e.g., rubella virus), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus); Poliovirus: I VP1 (Emini et al., 1983, Nature 304:699); Hepatitis B virus: hepatitis B surface antigen (Itoh et al., 1986, Nature 308:19; Neurath et al., 1986, Vaccine 4:34), hepatitis B virus core protein and/or hepatitis B virus surface antigen or a fragment or derivative thereof (see, e.g., U.K. Patent Publication No. GB 2034323A published Jun. 4, 1980; Ganem and Varmus, 1987, Ann. Rev. Biochem. 56:651-693; Tiollais et al., 1985, Nature 317:489-495), hepatitis (Hep B Surface Antigen (gp27S, gp36S, gp42S, p22c, pol, x)). Additional viruses include Ebola, Marburg, Rabies, Hanta virus infection, West Nile virus, SARS-like Coronaviruses, Varicella-zoster virus, Epstein-Barr virus, Alpha viruse, St. Louis encephalitis. Adenovirdiae (mastadenovirus and aviadenovirus), Leviviridae (levivirus, enterobacteria phase MS2, allolevirus), Poxyiridae (e.g., chordopoxyirinae, parapoxvirus, avipoxvirus, capripoxvirus, leporipoxvirus, suipoxvirus, molluscipoxvirus, and entomopoxyirinae), Papovaviridae (polyomavirus and papillomavirus); Paramyxoviridae (paramyxovirus, parainfluenza virus 1), Mobillivirus (measles virus), Rubulavirus (mumps virus), metapneumovirus (e.g., avian pneumovirus and human metapneumovirus); Pseudorabies: pseudorabies virus g50 (gpD), pseudorabies virus II (gpB), pseudorabies virus gIII (gpC), pseudorabies virus glycoprotein H, pseudorabies virus glycoprotein E; transmissible gastroenteritis including transmissible gastroenteritis glycoprotein 195, transmissible gastroenteritis matrix protein; Newcastle virus including Newcastle disease virus hemagglutinin-neuraminidase; infectious laryngotracheitis virus including viral antigens such as infectious laryngotracheitis virus glycoprotein G or glycoprotein 1; La Crosse virus including viral antigen such as a glycoprotein of La Crosse virus (Gonzales-Scarano et al., 1982, Virology 120:42),

Exemplary swine viruses include swine rotavirus glycoprotein 38, swine parvovirus capsid protein, Serpulina hydodysenteriae protective antigen, bovine viral diarrhea glycoprotein 55, neonatal calf diarrhea virus (Matsuno and Inouye, 1983, Infection and Immunity 39:155), hog cholera virus, African swine fever virus, swine influenza including antigens such as swine flu hemagglutinin and swine flu neuraminidase.

Exemplary equine viruses include equine influenza virus or equine herpesvirus: equine influenza virus type A/Alaska 91 neuraminidase, equine influenza virus type A/Miami 63 neuraminidase, equine influenza virus type A/Kentucky 81 neuraminidase, equine herpesvirus type 1 glycoprotein B, and equine herpesvirus type 1 glycoprotein D, Venezuelan equine encephalomyelitis virus (Mathews and Roehrig, 1982, J. Immunol. 129:2763).

Exemplary cattle viruses include bovine respiratory syncytial virus or bovine parainfluenza virus: bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, and bovine parainfluenza virus type 3 hemagglutinin neuraminidase), bovine viral diarrhea virus glycoprotein 48 or glycoprotein 53, infectious bovine rhinotracheitis virus: infectious bovine rhinotracheitis virus glycoprotein E or glycoprotein G, foot and mouth disease virus, punta toro virus (Dalrymple et al., 1981, in Replication of Negative Strand Viruses, Bishop and Compans (eds.), Elsevier, N.Y., p. 167).

### A. Coronaviruses

In some embodiments, the antigen is an antigen of coronavirus. The coronaviruses (order Nidovirales, family Coronaviridae, and genus Coronavirus) are a diverse group of large, enveloped, positive-stranded RNA viruses that cause respiratory and enteric diseases in humans and other animals.

Coronaviruses typically have narrow host and can cause severe disease in many animals, and several viruses, including infectious bronchitis virus, feline infectious peritonitis virus, and transmissible gastroenteritis virus, are significant veterinary pathogens. Human coronaviruses (HCoVs) are found in both group 1 (HCoV-229E) and group 2 (HCoV-OC43) and are historically responsible for ~30% of mild upper respiratory tract illnesses.

At ~30,000 nucleotides, their genome is the largest found in any of the RNA viruses. There are three groups of coronaviruses; groups 1 and 2 contain mammalian viruses, while group 3 contains only avian viruses. Within each group, coronaviruses are classified into distinct species by host range, antigenic relationships, and genomic organization. The genomic organization is typical of coronaviruses, with the characteristic gene order (5′-replicase [rep], spike [S], envelope [E], membrane [M], nucleocapsid [N]-3′) and short untranslated regions at both termini. The SARS-CoV rep gene, which comprises approximately two-thirds of the genome, encodes two polyproteins (encoded by ORFla and ORF1b) that undergo co-translational proteolytic processing. There are four open reading frames (ORFs) downstream of rep that are predicted to encode the structural proteins, S, E, M, and N, which are common to all known coronaviruses.

### I. SARS-CoV-2

In some embodiments, the antigen is an antigen from a severe acute respiratory syndrome coronavirus-2 (SARS-CoV-2) betacoronavirus of the subgenus Sarbecovirus. SARS-CoV-2 viruses share approximately 79% genome sequence identity with the SARS-CoV virus identified in 2003. An exemplary nucleic acid sequence for the SARS-CoV-2 ORF1a/b gene is set forth in GenBank accession number MN908947.3. The genome organization of SARS-CoV-2 viruses is shared with other betacoronaviruses; six functional open reading frames (ORFs) are arranged in order from 5′ to 3′: replicase (ORF1a/ORF1b), spike (S), envelope (E), membrane (M) and nucleocapsid (N). In addition, seven putative ORFs encoding accessory proteins are interspersed between the structural genes.

In some preferred embodiments, the antigen includes one or more SARS-CoV-2 antigens targeting one or more structural (S, E, M, N) and non-structural (NSPs) SARS-CoV-2 proteins with selected epitopes in conserved regions of the SARS-CoV-2 genome. In further preferred embodiments, the antigen includes one or more CD8 T cell peptides targeting one or more structural (S, M, N) and non-structural (NSPs) SARS-CoV-2 proteins with selected epitopes in conserved regions of the SARS-CoV-2 genome, eliciting T cell mediate immune response specific to the one or more SARS-CoV-2 antigens. Exemplary antigens include, but are not limited to the peptides listed in Table 1, and/or variants thereof.

TABLE 1

| | |
|---|---|
| Target | Exemplary Epitope (single amino acid code) |
| Spike glycoprotein (RBD epitope) | KIADYNYKL |
| Spike glycoprotein | VVFLHVTYV |
| N protein | LALLLLDRL |
| M protein | GLMWLSYFI |
| Protein 3a | YLYALVYFL |
| NSP3 | LLSAGIFGA |
| NSP4 | FLLNKEMYL |
| NSP6 | SMWALIISV |
| NSP12 | NLIDSYFVV |
| NSP13 | KLSYGIATV |
| NSP14 | MMISAGFSL |
| NSP16 | YLNTLTLAV |

### 2. Bacterial Antigens

In some embodiments, the antigen is a bacterial antigen. Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia. In some embodiments, the antigenic or immunogenic protein fragment or epitope (or mRNA encoding such) is derived from a pathogenic bacteria such as Anthrax; Chlamydia: Chlamydia protease-like activity factor (CPAF), major outer membrane protein (MOMP); Mycobacteria; Legioniella: Legionella peptidoglycan-associated lipoprotein (PAL), mip, flagella, OmpS, hsp60, major secretory protein (MSP); Diptheria: diptheria toxin (Audibert et al., 1981, Nature 289:543); Streptococcus 24M epitope (Beachey, 1985, Adv. Exp. Med. Biol. 185:193); Gonococcus: gonococcal pilin (Rothbard and Schoolnik, 1985, Adv. Exp. Med. Biol. 185:247); Mycoplasm: mycoplasma hyopneumoniae; Mycobacterium tuberculosis: M. tuberculosis antigen 85A, 85B, MPT51, PPE44, mycobacterial 65-kDa heat shock protein (DNA-hsp65), 6-kDa early secretary antigenic target (ESAT-6); Salmonella typhi; Bacillus anthracis B. anthracis protective antigen (PA); Yersinia perstis: Y. pestis low calcium response protein V (LcrV), F1 and F1-V fusion protein; Francisella tularensis; Rickettsia typhi; Treponema pallidum; Salmonella: SpaO and H1a, outer membrane proteins (OMPs); and Pseudomonas: P.aeruginosa OMPs, PcrV, OprF, OprI, PilA and mutated ToxA.

### 3. Fungal Antigens

In some embodiments, the antigenic or immunogenic protein fragment or epitope is derived from a pathogenic fungus, including, but not limited to,

Coccidioides immitis: Coccidioides Ag2/Pra106, Prp2, phospholipase (P1b), alpha-mannosidase (Amn1), aspartyl protease, Gell;

Blastomyces dermatitidis: Blastomyces dermatitidis surface adhesin WI-1;

Cryptococcus neoformans: Cryptococcus neoformans GXM and its Peptide mimotopes, and mannoproteins, Cryptosporidiums surface proteins gp15 and gp40, Cp23 antigen, p23;

Candida spp. inclduing C. albicans. C. glabrata. C. parapsilosis. C. dubliniensis. C. krusei. and others;

Aspergillus species: Aspergillus Asp f 16, Asp f 2, Der p 1, and Fel d 1, rodlet A, PEP2, Aspergillus HSP90, 90-kDa catalase.

### 4. Protozoan Antigens

In some embodiments, the antigenic or immunogenic protein fragment or epitope is derived from a pathogenic protozoan. Exemplary protozoa or protozoan antigens include:
Plasmodium falciparum, Plasmodium vivax, Plasmodium ovale, Plasmodium malariae, Plasmodium apical membrane antigen 1 (AMA1), 25-kDa sexual-stage protein (Pfs25), erythrocyte membrane protein 1 (PfEMP1) circumsporozoite protein (CSP), Merozoite Surface Protein-1 (MSP1);
Leishmania species:, Leishmania cysteine proteinase type III (CPC)
Trypanosome species (African and American): T. pallidum outer membrane lipoproteins, Trypanosome beta-tubulin (STIB 806), microtubule-associate protein (MAP p15), cysteine proteases (CPs)
Cryptosporidiums;
isospora species;
Naegleria fowleri;
Acanthamoeba species;
Balamuthia mandrillaris;
Toxoplasma gondii, or
Pneumocystis carinii: Pneumocystis carinii major surface glycoprotein (MSG), p55 antigen;
Babesia
Schistosomiasis: Schistosomiasis mansoni Sm14, 21.7 and SmFim antigen, Tegument Protein Sm29, 26 kDa GST, Schistosoma japonicum, SjCTPI, SjC23, Sj22.7, or SjGST-32
Toxoplasmosis: gondii surface antigen 1 (TgSAG1), protease inhibitor-1 (TgPI-1), surface-associated proteins MIC2, MIC3, ROP2, GRA1-GRA7.

### 5. Cancer or Tumor Antigens

In some embodiments, the antigen is a cancer antigen or a nucleic acid or vector thereof encoding a cancer antigen. A cancer antigen is an antigen that is typically expressed preferentially by cancer cells (i.e., it is expressed at higher levels in cancer cells than on non-cancer cells; cancer-associated antigen) and in some instances it is expressed solely by cancer cells (cancer-specific antigen). Cancer antigen may be expressed within a cancer cell or on the surface of the cancer cell. Exemplary cancer antigens include tumor-associated antigens (TAAs), tumor specific antigens (TSAs), tissue-specific antigens, viral tumor antigens, cellular oncogene proteins, and/or tumor-associated differentiation antigens. These antigens can serve as targets for the host immune system and elicit responses which result in tumor destruction. This immune response is mediated primarily by lymphocytes; T cells in general and class I MHC-restricted cytotoxic T lymphocytes in particular play a central role in tumor rejection. Hellstrom, K. E., et al., (1969) Adv. Cancer Res. 12:167 223; Greenberg, P. D. (1991) in Advances in Immunology, vol. 49 (Dixon, D. J., ed.), pp. 281 355, Academic Press, Inc., Orlando, FL. The cloning of TAAs for cancer immunotherapy is described e.g. in Boon, T., et al., (1994) Annu. Rev. Immunol. 12:337 365; Brithcard, V., et al., (1993) J. Exp. Med. 178:489 495; Cox, A. L., et al., (1994) Science 264:716 719; Houghton, A. N. (1994) J. Exp. Med. 180:14; Pardoll, D. M. (1994) Nature 369:357 358; Kawakami, Y., et al., (1994) Proc. Natl. Acad. Sci. U.S.A. 91:3515 3519; Kawakami, Y., et al., (1994) Proc. Natl. Acad. Sci. U.S.A. 91:6458 6462.

In general, viral vaccines are believed to mediate tumor rejection by activating class I MHC-restricted T-cells, particularly cytotoxic T lymphocytes (CTLs). T-cell activation is often potentiated by providing a suitable immunomodulator, for example, a T-cell co-stimulatory factor such as those of the B7 gene family. See e.g., Greenberg, P. D. (1991) in Advances in Immunology, Vol. 49 (Dixon, D. J., ed.), pp. 281 355, Academic Press, Inc., Orlando, Fla.; Fox B. A. et al. (1990) J. Biol. Response Mod. 9:499 511. The use of vaccinia viruses for anti-tumor immunotherapy has been described in Hu, S. L., Hellstrom, I., and Hellstrom K. E. (1992) in Vaccines: New Approaches to Immunological Problems (R. W. Ellis, ed) pp. 327 343, Butterworth-Heinemann, Boston. Anti-tumor responses have been elicited using recombinant pox viruses expressing TAAs such as carcinoembryonic antigen (CEA) and prostate specific antigen (PSA). (Muraro, R., et al., (1985) Cancer Res. 4S:5769 5780); (Kantor, J., et al,. (1992) J. Natl. Cancer Inst. 84:1084 1091); (Robbins, P. F., et al. (1991) Cancer Res. 51:3657 3662) (Kantor, J., et al., (1992) Cancer Res. 52:6917 6925.) No toxicity with these vectors was observed. However, in all cases the vaccines were injected.

Cancer antigens include, but are not limited to, melanoma TAAs such as MART-1 (Kawakami et al., J. Exp. Med. 180:347-352, 1994), MAGE-1, MAGE-3, GP-100, (Kawakami et al., Proc. Nat′l. Acad. Sci. U.S.A. 91:6458-6462, 1994), tyrosinase (Brichard et al. J. Exp. Med. 178:489, 1993), TAAs such as MUC-1, MUC-2, MUC-3, MUC-4, MUC-18, the point mutated ras oncogene and the point mutated p53 oncogenes (pancreatic cancer), PSA (prostate cancer), c-erb/B2 (breast cancer), KS ¼ pan-carcinoma antigen (Perez and Walker, 1990, J. Immunol. 142:3662-3667; Bumal, 1988, Hybridoma 7(4):407-415), ovarian carcinoma antigen (CA125) (Yu et al., 1991, Cancer Res. 51(2):468-475), prostatic acid phosphate (Tailor et al., 1990, Nucl. Acids Res. 18(16):4928), prostate specific antigen (PSA) (Henttu and Vihko, 1989, Biochem. Biophys. Res. Comm. 160(2):903-910; Israeli et al., 1993, Cancer Res. 53:227-230), melanoma-associated antigen p97 (Estin et al., 1989, J. Natl. Cancer Instit. 81(6):445-446), melanoma antigen gp75 (Vijayasardahl et al., 1990, J. Exp. Med. 171(4):1375-1380), high molecular weight melanoma antigen (HMW-MAA) (Natali et al., 1987, Cancer 59:55-63; Mittelman et al., 1990, J. Clin. Invest. 86:2136-2144), prostate specific membrane antigen, carcinoembryonic antigen (CEA) (Foon et al., 1994, Proc. Am. Soc. Clin. Oncol. 13:294), polymorphic epithelial mucin antigen, human milk fat globule antigen, colorectal tumor-associated antigens such as TAG-72 (Yokata et al., 1992, Cancer Res. 52:3402-3408), CO17-1A (Ragnhammar et al., 1993, Int. J. Cancer 53:751-758); GICA 19-9 (Herlyn et al., 1982, J. Clin. Immunol. 2:135), CTA-1 and LEA, Burkitt’s lymphoma antigen-38.13, CD19 (Ghetie et al., 1994, Blood 83:1329-1336), human B-lymphoma antigen-CD20 (Reffet al., 1994, Blood 83:435-445), CD33 (Sgouros et al., 1993, J. Nucl. Med. 34:422-430), melanoma specific antigens such as ganglioside GD2 (Saleh et al., 1993, J. Immunol., 151, 3390-3398), ganglioside GD3 (Shitara et al., 1993, Cancer Immunol. Immunother. 36:373-380), ganglioside GM2 (Livingston et al., 1994, J. Clin. Oncol. 12:1036-1044), ganglioside GM3 (Hoon et al., 1993, Cancer Res. 53:5244-5250), tumor-specific transplantation type of cell-surface antigen (TSTA) such as virally-induced tumor antigens including T-antigen DNA tumor viruses and Envelope antigens of RNA tumor viruses, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46:3917-3923), antigens of fibrosarcoma, human leukemia T cell antigen-Gp37 (Bhattacharya-Chatterjee et al., 1988, J. of Immuno specifically. 141:1398-1403), neoglycoprotein, sphingolipids, breast cancer antigen such as EGFR (Epidermal growth factor receptor), HER2 antigen (p185^{HER2}), polymorphic epithelial mucin (PEM) (Hilkens et al., 1992, Trends in Bio. Chem. Sci. 17:359), malignant human lymphocyte antigen-APO-1 (Bernhard et al., 1989, Science 245:301-304), differentiation antigen (Feizi, 1985, Nature 314:53-57) such as I antigen found in fetal erythrocytes, primary endoderm, I antigen found in adult erythrocytes, preimplantation embryos, I(Ma) found in gastric adenocarcinomas, M18, M39 found in breast epithelium, SSEA-1 found in myeloid cells, VEP8, VEP9, Myl, VIM-D5, D₁₅₆₋₂₂ found in colorectal cancer, TRA-1-85 (blood group H), C14 found in colonic adenocarcinoma, F3 found in lung adenocarcinoma, AH6 found in gastric cancer, Y hapten, LeY found in embryonal carcinoma cells, TL5 (blood group A), EGF receptor found in A431 cells, E₁ series (blood group B) found in pancreatic cancer, FC10.2 found in embryonic carcinoma cells, gastric adenocarcinoma antigen, CO-514 (blood group Le^{a}) found in Adenocarcinoma, NS-10 found in adenocarcinomas, CO-43 (blood group Le^{b}), G49 found in EGF receptor of A431 cells, MH2 (blood group ALe^{b}/Le^{y}) found in colonic adenocarcinoma, 19.9 found in colon cancer, gastric cancer mucins, T_{5A7} found in myeloid cells, R₂₄ found in melanoma, 4.2, G_{D3}, D1.1, OFA-1, G_{M2}, OFA-2, G_{D2}, and M1:22:25:8 found in embryonal carcinoma cells, and SSEA-3 and SSEA-4 found in 4 to 8-cell stage embryos T cell receptor derived peptides from Cutaneous T cell Lymphoma (Edelson, 1998, The Cancer Journal 4:62), IL-1, IL-2, IL-3, IL-4, IL-5, IL-6, IL-7, IL-8, IL-9, IL-10, IL-11, IL-12, IFN-α, IFN-β, IFN-β 17 mutants, IFN-65, CD2, CD3, CD4, CD5, CD8, CD11a, CD11b, CD11c, CD16, CD18, CD21, CD28, CD32, CD34, CD35, CD40, CD44, CD45, CD54, CD56, OX40L, 4-1BBL, K2, K1, Pβ, Oα, Mα, Mβ2, Mβ1, Hepsin, Pim-1, LMP1, TAP2, LMP7, TAP1, TRP, Oβ, IAβ, IAα, IEβ, IEβ2, IEa, CYP21, C4B, CYP21P, C4A, Bf, C2, HSP, G7a/b, TNF-α, TNF-β, D, L, Qa, T1a, COL11A2, DPβ2, DPα2, DPβ1, DPα1, DNα, DMα, DMβ, LMP2, TAPi1, LMP7, DOβ, DQβ2, DQα2, DQβ3, DQβ1, DQα1, DRβ, DRα, G250, HSP-70, HLA-B, HLA-C, HLA-X, HLA-E, HLA-J, HLA-A, HLA-H, HLA-G, HLA-F, nerve growth factor, somatotropin, somatomedins, parathormone, FSH, LH, EGF, TSH, THS-releasing factor, HGH, GRHR, PDGF, IGF-I, IGF-II, TGF-β, GM-CSF, M-CSF, G-CSF1, erythropoietin, β-HCG, 4-N-acetylgalactosaminyltransferase, GM2, GD2, GD3, JADE, BAGE, GAGE, XAGE, MUC-3, MUC-4, MUC-18, ICAM-1, C-CAM, V-CAM, ELAM, NM23, EGFR, E-cadherin, N-CAM, LFA-3 (CD58), EpCAM, B7.1, DCC, UTAA, melanoma antigen p75, K19, HKer 8, pMel 17, TP10, tyrosinase related proteins 1 and 2, p97, p53, RB, APC, DCC, NF-1, NF-2, WT-1, MEN-I, MEN-II, BRCA1, VHL, FCC and MCC, ras, myc, neu, raf, erb, src, fms, jun, trk, ret, gsp, hst, bcl and abl,, HRF, MIRL, CR1, CR2, CR3, CR4, C3a/C4a receptor, C5a receptor, Epstein-Barr Virus antigens (EBNA), BZLF-1, BXLF-1, and Nuclear Matrix Proteins, modified TAAs or TSAs, splice variants of TAAs or TSAs, functional epitopes, epitope agonists, and degenerate nucleic acid variations thereof.

In some embodiments the antigen is a neoantigen or a patient-specific antigen. Recent technological improvements have made it possible to identify the immune response to patient-specific neoantigens that arise as a consequence of tumor-specific mutations, and emerging data indicate that recognition of such neoantigens is a major factor in the activity of clinical immunotherapies (Schumacher and Schreidber, Science, 348(6230):69-74 (2015). Neoantigen load provides an avenue to selectively enhance T cell reactivity against this class of antigens.

Traditionally, cancer vaccines have targeted tumor-associated antigens (TAAs) which can be expressed not only on tumor cells but in the normal tissues (Ito, et al., Cancer Neoantigens: A Promising Source of Immunogens for Cancer Immunotherapy. J Clin Cell Immunol, 6:322 (2015) doi:10.4172/2155-9899.1000322). TAAs include cancer-testis antigens and differentiation antigens, and even though self-antigens have the benefit of being useful for diverse patients, expanded T cells with the high-affinity TCR (T-cell receptor) needed to overcome the central and peripheral tolerance of the host, which would impair anti-tumor T-cell activities and increase risks of autoimmune reactions.

Thus, in some embodiments, the antigen is recognized as “non-self” by the host immune system, and preferably can bypass central tolerance in the thymus. Examples include pathogen-associated antigens, mutated growth factor receptor, mutated K-ras, or idiotype-derived antigens. Somatic mutations in tumor genes, which usually accumulate tens to hundreds of fold during neoplastic transformation, could occur in protein-coding regions. Whether missense or frameshift, every mutation has the potential to generate tumor-specific antigens. These mutant antigens can be referred to as “cancer neoantigens” Ito, et al., Cancer Neoantigens: A Promising Source of Immunogens for Cancer Immunotherapy. J Clin Cell Immunol, 6:322 (2015) doi:10.4172/2155-9899.1000322. Neoantigen-based cancer vaccines have the potential to induce more robust and specific anti-tumor T-cell responses compared with conventional shared-antigen-targeted vaccines. Recent developments in genomics and bioinformatics, including massively parallel sequencing (MPS) and epitope prediction algorithms, have provided a major breakthrough in identifying and selecting neoantigens.

### C. T Cell Antigen Vectors

In some embodiments, the T cell antigen is administered in the form of a vector, to deliver and/or express the T cell antigen within the recipient. Exemplary vectors include nucleic acid plasmids, cosmids, replicon RNA, as virus vectors, bacterial, eukaryotic and protozoan cell vectors, and synthetic virus-like particles (VLPs) or liposomes encapsulating nucleic acids. Exemplary viral vectors include lentivirus, retrovirus, adenovirus, Adeno-associated virus (AAV) and alphavirus. Preferably the antigen vector is not a replicating viral vector.

Typically, nucleic acid vectors encode one or more genes which express one or more T cell antigens. A preferred T cell antigen expressed within the recipient is a polypeptide antigen, such as an MHC class I polypeptide antigen, or a precursor of an MHC class I polypeptide antigen. An exemplary vector is a nucleic acid plasmid, including one or more genes encoding one or more T cell antigens. In some embodiments, one or more gene(s) encoding antigens may be operably linked to a promoter to express the inserted gene. Promoters are well known in the art and can readily be selected depending on the host and the cell type one wishes to target. In certain embodiments, enhancer elements can also be used in combination to increase the level of expression. In certain embodiments, inducible promoters, which are also well known in the art, may be used. In some embodiments, the promoter is modulated by an external factor or cue, allowing control of the level of polypeptide being produced by the vectors by activating that external factor or cue. For example, heat shock proteins are proteins encoded by genes in which the promoter is regulated by temperature. The promoter of the gene which encodes the metal-containing protein metallothionine is responsive to Cd+ ions. Incorporation of this promoter or another promoter influenced by external cues also makes it possible to regulate the production of the polypeptides comprising antigen.

In some embodiments, the nucleic acid encoding at least one gene of interest encoding, e.g. an antigen, is operably linked to an “inducible” promoter. Inducible systems allow careful regulation of gene expression. See, Miller and Whelan, Human Gene Therapy, 8:803-815 (1997). The phrase “inducible promoter” or “inducible system” as used herein includes systems wherein promoter activity can be regulated using an externally delivered agent. Such systems include, for example, systems using the lac repressor from E. coli as a transcription modulator to regulate transcription from lac operator-bearing mammalian cell promoters (Brown et al., Cell, 49:603-612, 1987); systems using the tetracycline repressor (tetR)(Gossen and Bujard, Proc. Natl. Acad. Sci. USA 89: 5547-5551, 1992; Yao et al., Human Gene Ther. 9:1939-1950, 1998; Shokelt et al., Proc. Natl. Acad. Sci. USA `92.6522-6526, 1995). Other such systems include FK506 dimer, VP16 or p65 using castradiol, RU486/mifepristone, diphenol muristerone or rapamycin. Another example is an ecdysone inducible system (see, e.g. Karns et al., MBC Biotechnology 1:11, 2001). Inducible systems are available, e.g., from Invitrogen, Clontech, and Ariad. Systems using a repressor with the operon are preferred. These promoters may be adapted by substituting portions of pox promoters for the mammalian promoter.

In some embodiments, a “transcriptional regulatory element” or “TRE” is introduced for regulation of the gene of interest. A TRE is a polynucleotide sequence, preferably a DNA sequence, that regulates transcription of an operably-linked polynucleotide sequence by an RNA polymerase to form RNA. A TRE increases transcription of an operably linked polynucleotide sequence in a host cell that allows the TRE to function. The TRE comprises an enhancer element and/or viral promoter element, which may or may not be derived from the same gene. The promoter and enhancer components of a TRE may be in any orientation and/or distance from the coding sequence of interest, and comprise multimers of the foregoing, as long as the desired, transcriptional activity is obtained.

In some embodiments, an “enhancer” for regulation of the gene of interest is provided. An enhancer is a polynucleotide sequence derived from a gene which increases transcription of a gene which is operably-linked to a promoter to an extent which is greater than the transcription activation effected by the promoter itself when operably-linked to the gene, i.e., it increases transcription from the promoter.

The activity of a regulatory element such as a TRE or an enhancer generally depends upon the presence of transcriptional regulatory factors and/or the absence of transcriptional regulatory inhibitors. Transcriptional activation can be measured in a number of ways known in the art, but is generally measured by detection and/or quantification of mRNA or the protein product of the coding sequence under control of (i.e., operatively linked to) the regulatory element. The regulatory element can be of varying lengths, and of varying sequence composition. By transcriptional activation, it is intended that transcription will be increased above basal levels in the target cell by at least about 2-fold, preferably at least about 5-fold, preferably at least about 10-fold, more preferably at least about 20-fold. More preferably at least about 50-fold, more preferably at least about 100-fold, even more preferably at least about 200-fold, even more preferably at least about 400- to about 500-fold, even more preferably, at least about 1000-fold. Basal levels are generally the level of activity, if any, in a non-target cell, or the level of activity (if any) of a reporter construct lacking the TRE or enhancer of interest as tested in a target cell type.

Certain point mutations within sequences of TREs decrease transcription factor binding and gene activation. One of skill in the art would recognize that some alterations of bases in and around known the transcription factor binding sites are more likely to negatively affect gene activation and cell-specificity, while alterations in bases which are not involved in transcription factor binding are not as likely to have such effects. Certain mutations also increase TRE activity. Testing of the effects of altering bases may be performed in vitro or in vivo by any method known in the art, such as mobility shift assays, or transfecting vectors containing these alterations in TRE functional and TRE non-functional cells. Additionally, one of skill in the art would recognize that point mutations and deletions can be made to a TRE sequence without altering the ability of the sequence to regulate transcription.

### D. Additional Adjuvants and Agents

In some embodiments, the composition including an intact, non-replicating or replication-impaired poxvirus and a T cell antigen is administered in combination with one or more additional molecules that enhance or induce a T cell-mediated immune response within the recipient. Exemplary molecules include cytokines and co-stimulatory molecules.

In some embodiments, the composition administered to the subject, or co-expressed within the subject further include of a co-stimulatory molecule, a growth factor, or a cytokine. Exemplary molecules include IL-1, IL-2, IL-7, IL-12, IL-15, IL-18, IL-23, IL-27, B7-2, B7-H3, CD40, CD40L, ICOS-ligand, OX-40L, 4-1BBL, GM-CSF, SCF, FGF, Fantigen-ligand, and CCR4. In some embodiments, the one or more additional molecules is administered to the subject before, at the same time, and after the poxvirus and T cell antigen is administered. In some embodiments, the one or more additional molecules are administered to the subject to the same site that the T cell antigen is administered, or at a distant site.

### 1. Immunostimulatory Molecules

In some embodiments, the compositions also include one or more cytokines or co-stimulatory molecules. In some embodiments, the one or more cytokines or co-stimulatory molecules is co-administered with the antigen and the replication-deficient or non-replicating viral vectors. In some embodiments, the one or more cytokines or co-stimulatory molecules is encoded within the replication-deficient or non-replicating viral vectors. In some embodiments, the one or more cytokines or co-stimulatory molecules is encoded in a separate vector. Exemplary cytokines or co-stimulatory molecules, such as interleukin (IL) (e.g., IL-2, IL-4, IL-10, IL-12), an interferon (IFN) (e.g., IFN-γ), granulocyte macrophage colony stimulating factor (GM-CSF) or an accessory molecule (e.g., ICAM-1) or co-stimulatory molecules, e.g., B7.1, B7.2, may be used as adjuvants.

In certain embodiments, one or more of cytokines, co-stimulatory and other immunomodulatory molecules can be co-administered via co-insertion of the genes encoding the molecules into the replication-deficient or non-replicating viral vectors or a second vector which is admixed with the recombinant virus expressing the antigen. Alternatively, one or more of cytokines, co-stimulatory and other immunomodulatory molecules can be administered separately at the same site or different site, or systemically to the host. It may be desirable to administer a substantially pure preparation of the immunomodulator to boost efficacy of the host immune response to the T cell antigen.

Examples of costimulatory molecules include, but are not limited to, B7-1, B7-2, ICAM-1, CD40, CD40L, LFA-3, CD72, OX40L (with or without OX40). Examples of cytokines and growth factors include but are not limited to: granulocyte macrophage-colony stimulating factor (GM-CSF), granulocyte-colony stimulating factor (G-CSF), macrophage-colony stimulating factor (M-CSF), tumor necrosis factors (TNFα and TNFβ), transforming growth factors (TGFα and TGFβ), insulin-like growth factors (IGF-I and IGF-II), growth hormone, interleukins 1 to 15 (IL-1 to IL-15), interferons α, β, γ (IFN-α IFN-β and IFN-γ), brain-derived neurotrophic factor, neurotrophins 3 and 4, hepatocyte growth factor, erythropoictin, EGF-like mitogens, TGF-like growth factors, PDGF-like growth factors, melanocyte growth factor, mammary-derived growth factor 1, prostate growth factors, cartilage-derived growth factor, chondrocyte growth factor, bone-derived growth factor, osteosarcoma-derived growth factor, glial growth-promoting factor, colostrum basic growth factor, endothelial cell growth factor, tumor angiogenesis factor, hematopoietic stem cell growth factor, B-cell stimulating factor 2, B-cell differentiation factor, leukemia-derived growth factor, myelomonocytic growth factor, macrophage-derived growth factor, macrophage-activating factor, erythroid-potentiating activity, keratinocyte growth factor, ciliary neurotrophic growth factor, Schwann cell-derived growth factor, vaccinia virus growth factor, bombyxin, neu differentiation factor, v-Sis, glial growth factor/acetylcholine receptor-inducing activity, transferrin, bombesin and bombesin-like peptides, angiotensin II, endothelin, atrial natriuretic factor (ANF) and ANF-like peptides, vasoactive intestinal peptide, RANTES, Bradykinin and related growth factors.

In some of the preferred embodiments, the co-stimulatory molecule, growth factor, adjuvant or cytokine is IL-1, IL-2, IL-4, IL-7, IL1-9, IL-12, IL-15, IL-18, IL-23, IL-27, IL-31, IL-33, B7-1, B7-2, B7-H3, LFA-3, B7-H3, CD40, CD40L, ICOS-ligand, OX-40L, 4-1BBL, GM-CSF, SCF, FGF, Fantigen-ligand, CCR4, QS-7, QS-17, QS-21, CpG oligonucleotides, ST-246, AS-04, LT R192G mutant, Montanide ISA 720, heat shock proteins, synthetic mycobacterial cordfactor (CAF01), Lipid A mimetics, Salmonella enterica serovar Typhimurium flagellin (FliC), Montanide 720, Levamisole (LMS), Imiquimod, Diphtheria Toxin, IMP321, AS02A, AS01B, AS15-SB, Alhydrogel, Montanide ISA, Aluminum hydroxide, MF59, ISCOMATRIX, MLPA, MPL and other TLR-4 ligands, MDP and other TLR-2 ligands, CpG and TLR9 ligands, imiquimod and othe TLR7 ligands, resiquimod and TLR8 ligands, AS02A, AS01B, Heat Liable Toxin LTK63 and LT-R192G. In some embodiments, poxviruses expressing B7-1, ICAM-1, and LFA-3, also known as TRICOM, are provided that induce activation of both CD4⁺ and CD8⁺ T cells. (U.S. Pat. No. 6,045,802; Hodge et al., J. Natl. Cancer Inst. 92: 1228-39 (2000); Hodge et al., Cancer Research 59: 5800-07 (1999)). OX40 is a primary co-stimulator of T cells that have encountered antigen, rather than naive T cells, and promotes T-cell expansion after T cell tolerance is induced. (Bansal-Pakal et al., Nature Med. 7: 907-12 (2001)). OX40L plays a role during T cell activation by a) sustaining the long-term proliferation of CD4⁺ and CD8⁺ T cells, b) enhancing the production of Th1 cytokines such as IL-2, IGN-γ, and TNF-α from both CD4⁺ and CD8⁺ T cells without changing IL-4 expression, c) protecting T cells from apoptosis. In certain embodiments, the combination of B7-1, ICAM-1, LFA-3, and OX40L enhances initial activation and then further potentiates sustained activation of naive and effector T cells. Adjuvants can also be administered. In one embodiment, one administers a poxvirus vector containing B7, LFA-3 and ICAM-1 in conjunction with a tumor associated antigen. In a further embodiment, the poxvirus also contains OX40L. Other useful adjuvants that can be administered separately from the poxvirus are, for example, RIBI Detox (Ribi Immunochemical), QS21 (Aquila), incomplete Freund’s adjuvant.

In some embodiments, the compositions including poxviruses and T cell antigens are administered in the form of a composition including one or more other pharmaceutically acceptable carriers, including any suitable diluent or excipient. Preferably, the pharmaceutically acceptable carrier does not itself induce a physiological response, e.g., an immune response nor result in any adverse or undesired side effects and/or does not result in undue toxicity. Pharmaceutically acceptable carriers include, but are not limited to, saline, buffered saline, dextrose, water, glycerol, sterile isotonic aqueous buffer, and combinations thereof. Additional examples of pharmaceutically acceptable carriers, diluents, and excipients are provided in Remington’s Pharmaceutical Sciences (Mack Pub. Co., N.J., current edition).

### E. Pharmaceutically Acceptable Carriers

In some embodiments, the compositions include one or more pharmaceutically acceptable carriers, or excipients, or preservatives. Pharmaceutically acceptable carriers include compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration. Pharmaceutically acceptable carriers include, but are not limited to, buffers, diluents, preservatives, binders, stabilizers, a mixture or polymer of sugars (lactose, sucrose, dextrose, etc.), salts, and combinations thereof.

The compositions may be administered in combination with one or more physiologically or pharmaceutically acceptable carriers, thickening agents, cosolvents, adhesives, antioxidants, buffers, viscosity and absorption enhancing agents and agents capable of adjusting osmolarity of the formulation. Proper formulation is dependent upon the route of administration chosen. If desired, the compositions may also contain minor amounts of nontoxic auxiliary substances such as wetting or emulsifying agents, dyes, pH buffering agents, or preservatives.

The formulation may also be in the form of a suspension or emulsion. In general, pharmaceutical compositions are provided including effective amounts of the composition, and optionally include pharmaceutically acceptable diluents, preservatives, solubilizers, emulsifiers, adjuvants and/or carriers. Such compositions include diluents such as sterile water, buffered saline of various buffer content (e.g., Tris-HCl, acetate, phosphate), pH and ionic strength; and optionally, additives such as anti-oxidants (e.g., ascorbic acid, sodium metabisulfite), and preservatives and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate.

### III. Methods of Use

Methods for inducing or stimulating a T cell mediated immune response to a T cell antigen in epithelial tissues of a human subject are provided. Methods for effectively generating one or more populations of effector memory T cells (T_{EM}), tissue resident memory T cells (T_{RM}), and central memory T cells (T_{CM}) against the select T cell antigen(s) are also described.

### A. Methods of Inducing a T Cell Mediated Immune Response

Methods for inducing or stimulating a T cell mediated immune response to a T cell antigen in epithelial tissues of a subject typically include administering to disrupted epithelial tissue of the subject intact, non-replicating or replication-impaired poxvirus, preferably in combination with a T cell antigen or a vector expressing the T cell antigen.

In some embodiments, the non-replicating or replication-impaired poxvirus and the T cell antigen or a vector expressing the T cell antigen are in the same admixture, or in separate admixtures. In some embodiments, the step of administering the non-replicating or replication-impaired poxvirus and the step of administering T cell antigen or a vector expressing the T cell antigen are carried out simultaneous or sequential at the same epithelial tissue site or at two different the epithelial tissue sites of the subject.

### B. Individuals to Be Treated

A subject in need of treatment is a subject having or at risk of having cancer or a subject having or at risk of having an infection (e.g., a subject having or at risk of contracting a viral, bacterial, fungal or protozoal infection).

A subject having cancer is a subject that has been diagnosed with cancer, but may be in remission. “Cancer” as used herein refers to an uncontrolled growth of cells which interferes with the normal functioning of the bodily organs and systems. A subject at risk of developing a cancer is one who has a higher than normal probability of developing cancer. These subjects include, for instance, subjects having a genetic abnormality, the presence of which has been demonstrated to have a correlative relation to a higher likelihood of developing a cancer. These subjects also include subjects exposed to cancer causing agents (i.e., carcinogens) such as tobacco, asbestos, or other chemical toxins, or subjects who have previously been treated for cancer and are in apparent remission.

A subject having an infection is a subject that has been exposed to an infectious microorganism and has acute or chronic detectable levels of the microorganism in his/her body or has signs and symptoms of the infectious microorganism. Methods of assessing and detecting infections in a subject are known by those of ordinary skill in the art. A subject at risk of having an infection is a subject that may be expected to come in contact with an infectious microorganism. Examples of such subjects are medical workers or those traveling to parts of the world where the incidence of infection is high. In some embodiments, the subject is at an elevated risk of an infection because the subject has one or more risk factors to have an infection. Examples of risk factors to have an infection include, for example, immunosuppression, immunocompromised, age, trauma, burns (e.g., thermal burns), surgery, foreign bodies, cancer, newborns especially newborns born prematurely. The degree of risk of an infection depends on the multitude and the severity or the magnitude of the risk factors that the subject has. Risk charts and prediction algorithms are available for assessing the risk of an infection in a subject based on the presence and severity of risk factors. Other methods of assessing the risk of an infection in a subject are known by those of ordinary skill in the art. In some embodiments, the subject who is at an elevated risk of an infection may be an apparently healthy subject. An apparently healthy subject is a subject who has no signs or symptoms of disease.

Examples of viruses that can be treated by the described methods, or for which the described methods confer protection, include, but are not limited to, HIV, influenza, dengue, Hepatitis A virus, Hepatitis B virus, Hepatitis C virus, Human papilloma virus, Ebola, Marburg, Rabies, Hanta virus infection, West Nile virus, SARS-like Coronaviruses, Herpes simplex virus (HSV1 and HSV2), Varicella-zoster virus, Epstein-Barr virus, Human herpesvirus 8, Alpha viruses, St. Louis encephalitis. In some embodiments, the viruses that can be treated by the described methods, or for which the described methods confer protection are coronaviruses. In a specific embodiment, the virus is SARS-CoV-2.

Other viruses that may be treated or for which the methods described herein confer protection include, but are not limited to, enteroviruses (including, but not limited to, viruses that the family picornaviridae, such as polio virus, Coxsackie virus, echo virus), rotaviruses, adenovirus, and hepatitis virus, such as hepatitis A, B, C D and E. Specific examples of viruses that have been found in humans include, but are not limited to, Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bunyaviridae (e.g., bunya viruses, phleboviruses and Nairo viruses); Arenaviridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papillomaviruses, polyoma viruses); Adenoviridae (most adenoviruses); cytomegalovirus (CMV); Poxviridae (variola viruses, vaccinia viruses, pox viruses); Iridoviridae (e.g., African swine fever virus); and other viruses acute laryngotracheobronchitis virus, Alphavirus, Kaposi’s sarcoma-associated herpesvirus, Newcastle disease virus, Nipah virus, Norwalk virus, Papillomavirus, parainfluenza virus, and avian influenza.

Bacterial infections or diseases that can be treated or prevented are caused by bacteria including, but not limited to, Mycobacterium tuberculosis, Salmonella typhi, Bacillus anthracis, Yersinia perstis, Francisella tularensis, Legionella, Chlamydia, Rickettsia typhi, and Treponema pallidum. Other bacteria that may be treated or for which the methods described herein confer protection include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include, but are not limited to, Helicobacter pyloris, Borelia burgdorferi, Mycobacteria sps (e.g. M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pertenue, Leptospira, and Actinomyces israelli.

Fungal diseases that can be treated or prevented using the poxviruses and methods described herein include, but are not limited to, Coccidioides immitis, Blastomyces dermatitidis, Cryptococcus neoformans, Candida albicans, Aspergillus species. Other fungi that may be treated or for which the methods described herein confer protection include, but are not limited to: Histoplasma capsulatum, Coccidioides immitis, and Chlamydia trachomatis.

Protozoal diseases or infections that can be treated or prevented unclude but are not limited to, Malaria (Plasmodium falciparum, Plasmodium vivax, Plasmodium ovale, Plasmodium malariae), Leishmania species, Trypanosome species (African and American), cryptosporidiums, isospora species, Naegleria fowleri, Acanthamoeba species, Balamuthia mandrillaris, Toxoplasma gondii, and Pneumocystis carinii.

Cancers or tumors which may be treated or prevented, but are not limited to, melanoma, cutaneous squamous cell carcinoma, basal cell carcinoma, breast cancer, prostate adenocarcinoma, prostatic intraepithelial neoplasia, squamous cell lung carcinoma, lung adenocarcinoma, small cell lung carcinoma, ovary cancer of epithelial origin, colorectal adenocarcinoma and leiomyosarcoma, stomach adenocarcinoma and leiomyosarcoma, hepatocellular carcinoma, cholangiocarcinoma, ductal adenocarcinomas of pancreas, endocrine pancreatic tumors, renal cell carcinoma, transitional cell carcinoma of kidney and bladder, bladder squamous cell carcinoma, papillary thyroid cancer, follicular thyroid cancer, brain cancers (astrocytoma, glioblastoma multiforme).

### C. Administration to Epithelial Tissue

Methods of administrating intact, non-replicating or replication-deficient poxvirus, in combination with a T cell antigen, to epithelial tissues of a subject are provided. Typically, compositions for inducing antigen-specific T cell mediated immunity including intact, non-replicating or replication-impaired poxvirus and one or more T cell antigens or mRNA encoding for T cell antigens are administered at the same time as the epithelial tissue is disrupted to induce antigen-specific T cell mediated immune responses to antigen in a subject. Suitable epithelial tissues include skin, lung, oral mucosa, mucosa of the gastrointestinal tract, and reproductive mucosa. A preferred epithelial tissue is skin tissue. In some embodiments, the compositions are administered to a mechanically disrupted epidermal/upper dermal tissue of the subject.

In some embodiments, the epithelial tissue is mechanically disrupted by a scarification needle, a needle, an abrader or microneedles. In some embodiments, the epithelial tissue is disrupted essentially at the same time as the administration of the poxvirus and the T cell antigen, or the vector expressing the T cell antigen. In other embodiments, the epithelial tissue is disrupted before administration of the poxvirus and the T cell antigen, or the vector expressing the T cell antigen. In some embodiments, the methods deliver the poxvirus to a first disrupted epithelial tissue location on the subject, and deliver the T cell antigen, or vector expressing the T cell antigen to a second disrupted epithelial tissue location of the subject. In some embodiments, the type of epithelial tissue of the first disrupted epithelial tissue location is different from that of the second disrupted epithelial tissue location.

Human beings can live for 70 years or more, spending much of that time free of symptomatic infectious diseases. The adaptive immune system, through the generation and maintenance of protective immunologic memory, permits people to live amongst a plethora of pathogens for many decades. The body has several epithelial surfaces that interface with the environment. The most accessible is skin, but continuous with skin is the oropharyngeal mucosal epithelium, the female reproductive epithelium, and the large and complex epithelial tissues that line the respiratory and gastrointestinal tracts. For pathogens to gain access to blood and internal tissues, they must infect and then breach one or more of these epithelial barriers. While each of these epithelial tissues is structurally different and employs different innate immune defenses, the adaptive immune system mediates lifelong protection against pathogen attack, through T cell memory and B cell antibody production.

Protective T cell memory is mediated by cells circulating in blood and through secondary lymphoid tissues. According to this view, while memory T cells could be readily mobilized from these compartments to peripheral epithelial tissues to captain the immunologic defense forces fighting infection, T cells were thought to return to blood and lymph nodes once the infection was resolved. Recently, memory T cells have been found in peripheral tissues, in abundance, in both humans and mice. Because of its accessibility, the majority of human work has been done in skin. Seminal work in this area has demonstrated that twice as many T cells reside in normal, non-inflamed human skin as in blood, and there are more than 20-fold more of skin homing memory T cells in normal skin than in blood. These skin resident T cells (T_{RM}) have a diverse T cell receptor repertoire, can be readily activated through the TCR or by cytokines, and have great proliferative potential. Moreover, they are polyfunctional with regard to cytokine production, suggesting their authentic role as memory T cells that protect against infection. Similar populations of T_{RM} exist in lung and GI tract, as well as reproductive mucosa. T cells are recruited to skin and other epithelial tissues after pathogen challenge, and then can persist there long term. At least two separate studies examining viral infection of skin have demonstrated that these T_{RM}, rather than antibody or T_{CM} recruited from blood, provide principal protection against viral challenge, even many months after the initial infection (Liu et al., Nature Medicine. 2010 Feb;16(2):224-7. Epub 2010 Jan 17). Similar populations of T cells have been identified in lung (Connor et al., Eur J Immunol. 2010 Sep;40(9):2482-92) and GI tract (Masopust et al. J Exp Med. 2010 Mar 15;207(3):553-64. Epub 2010 Feb 15) after infection. These resident T_{EM}/T_{RM} consist largely of T cells that were originally (as naïve T cells) activated in lymph nodes draining that tissue (Liu et al., Immunity. 2006;25(3):511-20), and thus represent a resident army of tissue specific T cells specific for tissue selective pathogens.

T cell recruitment into extranodal peripheral tissues is a highly regulated process controlled by the sequential interactions of adhesion molecules and chemokine receptors that are differentially expressed on various T cell subsets and their target. Human skin-homing T cells express cutaneous lymphocyte antigen (CLA) which binds to skin microvasculature-expressed E-selectin, typically in combination with CCR4 whose ligand CCL17 (TARC) is constitutively expressed on skin endothelium. Gut-homing T cells express α4β7 integrin and chemokine receptor CCR9, to which the corresponding ligands MadCAM-1 and CCL25 (TECK) are expressed on the endothelium and epithelium of the small intestine. Following VACV skin scarification, the skin homing molecules E- and P-selectin ligands (E-lig and P-lig, the functional murine equivalents of human CLA) are strongly upregulated on antigen-specific CD8 T cells between the 3^{rd} and 10^{th} cell divisions in the regional LN draining the scarified site. Subsets of proliferating VV specific CD8 T cells leave the draining inguinal node after as few as three cell divisions and migrate through blood to skin (T_{EM}), or to other LN (T_{CM}), respectively. In these distant LN, vaccinia-specific T_{CM} cells continue to proliferate, in the absence of continued antigen receptor stimulation, and acquire homing receptors consistent with the regional drainage of the LN they have migrated to, e.g., α4β7-integrin in mesenteric LN. Thus, generalized CD8 T cell mediated immunity to a local challenge is acquired by systemic dissemination of activated T cells from the local draining LN. The tissue homing properties of these cells then are imprinted in the respective LN environments to which they disseminate.

Modes of vaccine delivery play a critical role in the generation of T_{RM}. Different modes of vaccine delivery can generate similar levels of antibody and T_{CM}, but vastly different levels of T_{RM}. Intranasal vaccine generates a robust population of lung resident memory T cells (T_{RM}), but relatively few skin, liver, or GI tract T_{RM}. Intraperitoneal administration does not generate large populations of skin or lung T_{RM}. Intramuscular administration is ineffective at generating T_{RM} in most tissues. The presence or absence of T_{RM} is reflected in resistance to infection, and demonstrates clearly that simply sampling blood for antibody titers and/or memory T cell abundance and function as a surrogate for protective immunity falls far short of this goal and may even be misleading.

Skin scarification with vaccinia virus (VACV) is far and away the most effective way of generating both optimal T_{RM} populations as well as T_{CM}, not only in skin, but also in distant epithelial tissues. Mimicking infection through an epithelial tissue is essential to generating the robust protective immunity that the vertebrate immune system has, over millions of years, evolved to generate. Skin scarification replicates how pathogens breach the barrier of skin to infect it. From this perspective, it seems particularly irrational to vaccinate through skeletal muscle. While epithelial tissues are rich in dendritic cells and stromal cells that modify immune response, and lymph nodes draining these tissues have been shown to be elite “training academies” for T cells, muscle is not physiologically exposed to pathogens (or danger signals), does not contain abundant dendritic cells, and is drained by deeper LN that are unable to instruct T cells to traffic to peripheral tissues. While a tried and true means of generating neutralizing antibody responses, intramuscular vaccination is a woefully ineffective means of generating T_{RM}.

Significant populations of T_{RM} can be readily demonstrated in human skin. Normal skin of healthy adults contains approximately 20 billion memory T cells, nearly twice the number of T cells that are present in the entire circulation. Moreover, when the relevant skin homing T cell subset was considered, nearly 98% of T cells with a skin homing phenotype were found in skin rather than blood under normal, non-inflamed conditions. These skin homing/resident T cells are all CD45RO⁺ memory cells, predominantly αβ TCR⁺, CLA⁺ CCR4⁺, and express significant levels of CCR6, somewhat less CCR8 and CXCR6, and a subset of about 15-20% express both CD62L and CCR7. Thus, skin T cells are about 60% T_{RM}, 20% T_{MM} and 20% T_{CM}. They contain significant numbers of TNFα and IL-17 producing T cells, as well as T cells that produce IFN-γ; relatively few cells that produce IL-4 are present. TCR Vβ antibody FACS analysis indicates that these T_{RM} are highly diverse, and all Vβ families are represented. Nearly half of these T_{RM} expressed the activation markers CD25 (at intermediate levels) and CD69. T_{RM} are also much more responsive to stimulation than cells from the blood.

An analogous population of T_{RM} resides in human lung. These tissue resident cells are also exclusively CD45RO+, and do not bear either skin (CLA/CCR4 co expression) or gut (α4β7 integrin) homing markers, but express integrin α1β1. Their expression of CD69 (>50% positive) indicates that they are true tissue resident cells, and not simply cells that are trapped in the pulmonary vasculature (peripheral blood T cells are uniformly negative for CD69, while T_{RM} in humans and mice express significant CD69)). Based on similar cell counting methodologies as used in skin, it has been calculated that there are roughly 10 billion T cells in lung, half as many as the total number of T_{cells} in skin. While resident T cells in skin can be long lived and accumulate over time, recent work has shown that lung TRM are short lived, and each population generated will decay rapidly with a half life of ~5 days. An intravascular population in lung may fulfill the rapid response role of TRM in tissue. Large resident T cell populations are also found in human large and small bowel.

Repetitive encounter with antigen through a peripheral tissue generates increasing numbers of T_{RM} that accumulate in peripheral tissues over months, years, and decades in humans. The distribution of T_{RM} after immunization can be modified by administering mediators that have been shown to alter the expression of T cell homing markers. For example, retinoic acid has been shown to enhance the expression of gut homing markers, while reducing skin homing markers.

Antigens from a pathogen encountered through infected skin are presented by skin-derived dendritic cells in lymph nodes draining skin, and activate naïve T cells to proliferate and force their differentiation into skin homing T cells as well as central memory T cells. These skin homing T cells rapidly traffic to skin, seeding both infected skin and normal skin. A subset of these T cells then takes up residence in skin for years to decades. This occurs repeatedly throughout childhood, adolescence, and adulthood. For antigens encountered multiple times over the years, an amplified population of central memory T cells can be activated in lymph node, and these in turn can differentiate into skin homing T cells and seed skin in even greater numbers. One expects that the skin contains polyfunctional CD45RO+/ CLA+/CCR4+ T_{RM} enriched for common skin pathogens, such as C albicans, S aureus, Trichophyton species, P.acnes, P.ovale, HSV, as well as VACV and Yellow Fever (in vaccinated individuals), but not for common gut or lung pathogens. The frequency of T_{RM} responsive to these microorganisms will vary from individual to individual, but should be significantly higher than the frequency of responsive T_{CM} in peripheral blood, and even higher than the frequency of responsive naïve T cells.

The lung should contain polyfunctional CD45RO+/VLA-1+ T_{RM} enriched for common lung pathogens, for example, Influenza virus, M pneumonia, S.pneumonia, Adenovirus species, Rhinovirus species. The gastrointestinal tract should contain polyfunctional CD45RO/α4β7+ T_{RM} enriched for those specific for rotavirus, norovirus, E.coli, enteroviruses, H. pylori, calciviruses.

In contrast, the blood contains T_{CM} and T_{EM} specific for all antigens found in skin, lung, and colon, as well as endogenous viruses such as CMV and EBV. Most antigen reactive T cells will be non-polyfunctional TCM. To maximize immune responses using the route of administration described herein, i.e., mechanical disruption of the epithelial tissue at the time of exposure to replication deficient virus expressing one or more exogenous antigen, vaccines to infectious diseases would have to be evaluated not based solely on their ability to generate a neutralizing antibody response, but rather their capacity to generate populations of antigen specific T_{EM} and T_{CM} that would migrate to relevant tissues and become T_{RM}.

Vaccines to prevent the emergence or recurrence of human cancers would have to be similarly be modified so as to generate optimal populations of T_{RM} that would be deployed to the relevant tissue. For example, the HPV vaccine for cervical cancer might be modified to deliver T_{RM} to reproductive mucosa.

Immunologically mediated diseases that involve peripheral tissues, including skin (e.g., psoriasis), lung (e.g. asthma), and gut (e.g., inflammatory bowel disease) would have to be studied to fully establish the role of T_{RM} in these processes. The implication of T_{RM} playing a major role in disease activity, or the tendency for these diseases to chronically recur, would lead to treatment strategies that might be very different from those currently employed.

Intact, non-replicating or replication-deficient poxvirus, in combination with a T cell antigen are preferably administered by mechanical disruption of the epidermis (e.g., by skin scarification, scratching, abrading or superficial cutting). Typically, epithelial skin tissue is disrupted without penetrating the entire epidermis, and non-replicating or replication-impaired poxvirus and T cell antigen, or vector expressing the T cell antigen are administered to the stratum corneum.

Methods and devices for disrupting the skin and for depositing a substance into the epidermis of the skin are known in the art. Examples of devices for disrupting the skin include a scarification needle, a hypodermic needle, or an abrader. In one embodiment, the device is incorporated as part of a sterile vial containing lyophilized vaccine, which is rehydrated either from a separate source of diluent or by perforating a membrane separating the vaccine from the diluent.

The device mechanically disrupts the epidermis (e.g., abrader) by moving or rubbing over the epidermal tissue such as the skin. Alternatively, a chemical abrader is used, such as a surfactant to disrupt the mucosal layer in the lung, gastrointestinal tract or reproductive tract. It is preferred that the minimum amount of abrasion/mechanical disruption to produce the desired result be used. Determination of the appropriate amount of abrasion/mechanical disruption for a selected poxvirus alone or in combination with a T cell antigen, and/or formulation thereof is within the ordinary skill in the art. In another embodiment, one or more of the composition is applied in dry form to the abrading surface of the abrading device prior to application. In this embodiment, a reconstituting liquid is applied to the skin at the delivery site and the poxvirus and/or T cell antigen-coated abrading device is applied to the skin at the site of the reconstituting liquid. It is then moved or rubbed over the skin so that the poxvirus and/or T cell antigen becomes dissolved in the reconstituting liquid on the surface of the skin and is delivered simultaneously with abrasion. Alternatively, a reconstituting liquid may be contained in the device (e.g., a scarification needle, a hypodermic needle, or an abrader) and released to dissolve the disclosed composition as the device is applied to the skin for mechanical disruption of the epidermis. Certain disclosed composition may also be coated on the device (e.g., abrading device) in the form of a gel.

The devices are preferably capable of disrupting the skin to penetrate the epidermis without reaching and/or penetrating the dermis. In some embodiments, the device penetrates the stratum corneum without penetrating the entire epidermis.

In some embodiments, the compositions are administered to one or more specific layers of the skin. For example, in some embodiments, the compositions are administered to the epidermis. Suitable layers of the epidermis include the stratum corneum, stratum lucidum, stratum granulosum, stratum pinosum, and stratum basale. In other embodiments, the compositions are administered to the dermis. In other embodiments, the compositions are administered to the subcutis, or to a combination of the epidermis, dermis and/or subcutis.

In certain embodiments, the composition to be administered using the described methods are applied to the skin prior to abrading, simultaneous with abrading, or post-abrading.

In order to achieve the desired mechanical disruptions of the epidermis, the device should be moved across a subject’s skin at least once. The subject’s skin may be disrupted in alternating directions. The surface of the device may be coated with the disclosed composition. The length and thickness of the microprotrusions are selected based on the particular substance being administered and the thickness of the epidermis in the location where the device is to be applied. The microprotrusions penetrate the epidermis without piercing or passing through the entire dermis. In some embodiments, the protrusions penetrate the stratum corneum substantially without piercing or passing through the entire epidermis.

In some embodiments, the device is moved about 2 to 15 centimeters (cm). In some embodiments, the device is moved to produce a mechanically disrupted epidermal site having a surface area of about 4 cm² to about 300 cm². The extent of the mechanical disruption of the epidermis is dependent on the pressure applied during movement and the number of repetitions with the device.

In some embodiments, devices such as a scarification needle or an abrader for accurately targeting the epidermal space are provided. These devices may have solid or hollow microprotrusions. The microprotrusions can have a length up to about 1500 microns. In some embodiments, the microprotrusions have a length of about 200 to 1500 microns. In some embodiments, the microprotrusions have a length of about 300 to 1000 microns, or in the range of about 400 to 800 microns. Microneedle devices for administration of vaccine are available from BioSeren Tach Inc and described in U.S. Pat. No. 6,334,856. Skin patches are described in U.S. Pat. No. 6,706,693. These devices can be made of a biocompatible polymer or metal.

Any device known in the art for disruption of the epidermis by mechanical disruption can be used in the methods described herein. These include for example, microelectromechanical (MEMS) devices with arrays of short microneedles or microprotrusions, sandpaper-like devices, scrapers, and scarification needles.

### D. Combination Therapies

In some embodiments, the compositions are administered in further combination with one or more additional therapeutic agents. The agents can be administered in the same or separate pharmaceutical composition from the poxvirus, antigen, adjuvant, or combinations thereof.

In some embodiments, the compositions are administered in combination with a conventional therapeutic agent used for treatment of the disease or condition being treated. Conventional therapeutics agents are known in the art and can be determined by one of skill in the art based on the disease or disorder to be treated. In some embodiments, the method(s) for treating or preventing cancer described herein may be used in combination with one or more anticancer agents such as conventional chemotherapeutic agents. In some embodiments, the method(s) for treating or preventing infections is used in combination with one or more anti-bacterial agents, anti-viral agents, anti-fungal agents, or anti-protozoal agents. When administered as a cancer vaccine, the disclosed compositions may be administered in combination with a checkpoint inhibitor (PD1, CTLA4, TIM3, etc.).

### E. Dosage and Treatment Regimens

### 1. Dosage and Effective Amounts

Dosage units of reagents for providing a T cell-mediated immune response to an antigen in a subject by mechanical disruption of an epidermal tissue are also provided. In some embodiments, the dosage units include an effective amount for inducing or stimulating a protective T cell mediated immune response to a T cell antigen in epithelial tissues such as skin, lung, oral mucosa, gastrointestinal tract, and reproductive mucosa comprising an intact, non-replicating or replication-impaired poxvirus and a T cell antigen, or vector expressing the T cell antigen.

The compositions are typically administered to a subject in need thereof in an effective amount. For example, the compositions can be administered in a dosage sufficient reduce or prevent one or more symptoms of the cancer or an infectious disease, or to otherwise provide a desired pharmacologic and/or physiologic effect. The symptom may be physical or biological. For example, in the context of cancer treatment, the symptom may be physical, such as tumor burden, or biological such as proliferation of cancer cells. In some embodiments, the amount is effective to increase the killing of tumor cells or inhibit proliferation or metastasis of tumor cells. In some embodiments, the amount is effective to reduce tumor burden. In some embodiments, the amount is effective to reduce or prevent at least one comorbidity of a cancer or infection.

The precise dosage will vary according to a variety of factors such as subject-dependent variables (e.g., age, immune system health, clinical symptoms etc.). Persons of ordinary skill can determine optimum dosages, dosing methodologies and repetition rates, which may vary depending on the relative potency of individual vaccines, and can generally be estimated based on EC50s found to be effective in ex vivo assay and in in vivo animal models.

In some embodiments, an immune response to the antigen can be generated by administering between about 100-fold to about a 100-fold less pfu (plaque forming units) of the non-replicating or replication-impaired poxvirus along with a T cell antigen or vector containing the T cell antigen, when applied by mechanical disruption of the epidermis compared to conventional injection routes. In certain embodiments, a specific immune response to the antigen can be generated by administering between about 90-fold, 80-fold, 70-fold, 60-fold, 50-fold, 40-fold, 30-fold, 20-fold, 10-fold, 5-fold less pfu of the non-replicating or replication-impaired poxvirus when applied by mechanical disruption of the epidermis compared to conventional injection routes. In some embodiments a single deposition of the non-replicating or replication-impaired poxvirus and a T cell antigen is required to elicit a long-lasting, potent antigen-specific immune response in the subject.

In some embodiments, the compositions are administered on a dosage schedule, for example, an initial administration followed by one or more subsequent booster administrations. Thus in some embodiments, the composition is administered 2, 3, 4, or more times, for example, 1, 2, 3, 4, 5, 6, 7, 8, 9, 10, 11, 12, 13, 14, or 15 days, weeks, months, or years apart. Dosage regimens or cycles of the compositions and/or additional therapeutic agents can be completely or partially overlapping, or can be sequential. In particular embodiments, a second dose of the compositions is administered anywhere from two weeks to one year, preferably from one to six months, after the initial administration. Additionally, a third dose may be administered after the second dose and from three months to two years, or even longer, preferably 4 to 6 months, or 6 months to one year after the initial administration. The boosting antigen may be administered using the same format as the initial dose, or as a whole protein, an immunogenic peptide fraction of the protein, another recombinant viral vector, or DNA or mRNA encoding the protein or peptide. In some embodiments, different format of T cell antigens are used. For example, vaccinia may be followed by an avipox such as fowlpox, or vice versa. In some preferred embodiments, no booster immunization is required. In some embodiments, the intact, non-replicating or replication-deficient poxvirus is only given with the initial administration. In other embodiments, the intact, non-replicating or replication-deficient poxvirus is only given with one or more subsequent booster administrations.

### 2. Controls

The therapeutic result of the compositions including an intact, non-replicating or replication-deficient poxvirus and a T cell antigen can be compared to a control. Suitable controls are known in the art and include, for example, untreated cells or an untreated subject. A typical control is a comparison of a condition or symptom of a subject prior to and after administration of the targeted agent. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art.

### F. Methods for Determining Immune Responses

Methods for determining immune responses are known in the art. In some embodiments, viral lesions can be examined to determine the occurrence of an immune response to the virus and/or the antigen. In some embodiments, in vitro assays may be used to determine the occurrence of an immune response. Examples of such in vitro assays include ELISA assays and cytotoxic T cell (CTL) assays. In some embodiments, the immune response is measured by detecting and/or quantifying the relative amount of an antibody, which specifically recognizes an antigen in the sera of a subject who has been treated by administering the live, modified, non-replicating or replication-impaired poxvirus comprising the antigen, relative to the amount of the antibody in an untreated subject.

Techniques for the assaying antibodies and antibody filters in a sample are known in the art and include, for example, sandwich assays, ELISA and ELISpot. Polyclonal sera are relatively easily prepared by injection of a suitable laboratory animal with an effective amount of the immune effector, or antigenic part thereof, collecting serum from the animal and isolating specific sera by any of the known immunoadsorbent techniques. Antibodies produced by this method are utilizable in virtually any type of immunoassay.

The use of monoclonal antibodies in an immunoassay is preferred because of the ability to produce them in large quantities and the homogeneity of the product. The preparation of hybridoma cell lines for monoclonal antibody production derived by fusing an immortal cell line and lymphocytes sensitized against the immunogenic preparation can be achieved by techniques which are well known to those who are skilled in the art. In other embodiments, ELISA assays may be used to determine the level of isotype specific antibodies using methods known in the art.

Cytotoxic T cell (CTL) assays can be used to determine the lytic activity of CTLs, measuring specific lysis of target cells expressing a certain antigen. Immune-assays may be used to measure the activation (e.g., degree of activation) of sample immune cells. Sample immune cells refer to immune cells contained in samples from any source, including from a human patient, human donor, animal, or tissue cultured cell line. The immune cell sample can be derived from peripheral blood, lymph nodes, bone marrow, thymus, any other tissue source including in situ or excised tumor, or from tissue or organ cultures. The sample may be fractionated or purified to generate or enrich a particular immune cell subset before analysis. The immune cells can be separated and isolated from their source by standard techniques.

Immune cells include both non-resting and resting cells, and cells of the immune system that may be assayed, including, but not limited to, B lymphocytes, T lymphocytes, natural killer (NK) cells, lymphokine-activated killer (LAK) cells, monocytes, macrophages, neutrophils, granulocytes, mast cells, platelets, Langerhans cells, stem cells, dendritic cells, and peripheral blood mononuclear cells.

Immune cell activity that may be measured include, but is not limited to (1) cell proliferation by measuring the cell or DNA replication; (2) enhanced cytokine production, including specific measurements for cytokines, such as γIFN, GM-CSF, or TNF-alpha, IFN-alpha, IL-6, IL-10, IL-12; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; (9) chemokine secretion such as IP-10; (10) expression of costimulatory molecules (e.g., CD80, CD 86) and maturation molecules (e.g., CD83), (12) upregulation of class II MHC expression; and (13) apoptosis, which refers to fragmentation of activated immune cells under certain circumstances, as an indication of abnormal activation.

High throughput DNA sequencing of alpha and beta chains of the T cell receptor is another way of measuring a T cell response, as every naïve T cell (and every clone of memory T cells derived from that cells) has a unique CDR3 DNA sequence in each receptor chain. This can measure both the quantity/number and the diversity of T cells in a tissue.

Reporter molecules may be used for many of the immune assays described. A reporter molecule is a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i. e. radioisotopes) and chemiluminescent molecules. In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta- galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-antigen complex, allowed to bind, and then the excess reagent is washed away. A solution containing the appropriate substrate is then added to the complex of antibody-antigen- antibody. The substrate will react with the enzyme linked to the second antibody, giving a qualitative visual signal, which may be further quantitated, usually spectrophotometrically, to give an indication of the amount of antigen which was present in the sample. Alternately, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic colour visually detectable with a light microscope. The fluorescent labeled antibody is allowed to bind to the first antibody-antigen complex. After washing off the unbound reagent, the remaining tertiary complex is then exposed to the light of the appropriate wavelength the fluorescence observed indicates the presence of the antigen of interest.

Examples of some common immune assays are:

Cell Proliferation Assay: Activated immune cell proliferation is intended to include increase in cell number, cell growth, cell division, or cell expansion, as measured by cell number, cell weight, or by incorporation of radiolabeled nucleic acids, amino acids, proteins, or other precursor molecules. As one example, DNA replication is measured by incorporation of radioisotope labels. In some embodiments, cultures of stimulated immune cells can be measured by DNA synthesis by pulse-labeling the cultures with tritiated thymidine (³H-Tdr), a nucleoside precursor that is incorporated into newly synthesized DNA. Thymidine incorporation provides a quantitative measure of the rate of DNA synthesis, which is usually directly proportional to the rate of cell division. The amount of ³H-labeled thymidine incorporated into the replicating DNA of cultured cells is determined by scintillation counting in a liquid scintillation spectrophotometer. Scintillation counting yields data in counts per minute (cpm) which may then be used as a standard measure of immune cell responsiveness. The cpm in resting immune cell cultures may be either subtracted from or divided into cpm of the primed immune cells, which will yield a stimulation index ratio.

Flow cytometry can also be used to measure proliferation by measuring DNA with light scatter, Coulter volume and fluorescence, all of which are techniques that are well known in the art.

Enhanced Cytokine Production Assay: A measure of immune cell stimulation is the ability of the cells to secrete cytokines, lymphokines, or other growth factors. Cytokine production, including specific measurements for cytokines, such as γIFN, GM-CSF, or TNF-alpha, may be made by radioimmunoassay (RIA), enzyme-linked immunoabsorbent assay (ELISA), bioassay, or measurement of messenger RNA levels. In general, with these immunoassays, a monoclonal antibody to the cytokine to be measured is used to specifically bind to and thus identify the cytokine. Immunoassays are well known in the art and can include both competitive assays and immunometric assays, such as forward sandwich immunoassays, reverse sandwich immunoassays and simultaneous immunoassays.

In each of the above assays, the sample-containing cytokine is incubated with the cytokine-specific monoclonal antibody under conditions and for a period of time sufficient to allow the cytokines to bind to the monoclonal antibodies. In general, it is desirable to provide incubation conditions sufficient to bind as much cytokine and antibody as possible, since this will maximize the signal. Of course, the specific concentrations of antibodies, the temperature and time of incubation, as well as other such assay conditions, can be varied, depending upon various factors including the concentration of cytokine in the sample, the nature of the sample, and the like. Those skilled in the art will be able to determine operative and optimal assay conditions for each determination by employing routine experimentation.

Cell-Mediated Target Cell Lysis Assay: Another type of indicator for degree of immune cell activation is immune cell-mediated target cell lysis, which is meant to encompass any type of cell killing, including cytotoxic T lymphocyte activity, apoptosis, and the induction of target lysis by molecules secreted from non-resting immune cells stimulated to activity. Cell-mediated lympholysis techniques typically measure the ability of the stimulated immune cells to lyse ⁵¹Cr-labeled target cells. Cytotoxicity is measured as a percentage of ⁵¹Cr released in specific target cells compared to percentage of ⁵¹Cr released from control target cells. Cell killing may also be measured by counting the number of target cells, or by quantifying an inhibition of target cell growth.

Cell Differentiation Assay: Another indicator of immune cell activity is immune cell differentiation and maturation. Cell differentiation may be assessed in several different ways. One such method is by measuring cell phenotypes. The phenotypes of immune cells and any phenotypic changes can be evaluated by flow cytometry after immunofluorescent staining using monoclonal antibodies that will bind membrane proteins characteristic of various immune cell types.

A second means of assessing cell differentiation is by measuring cell function. This may be done biochemically, by measuring the expression of enzymes, mRNA’s, genes, proteins, or other metabolites within the cell, or secreted from the cell. Bioassays may also be used to measure functional cell differentiation.

Immune cells express a variety of cell surface molecules which can be detected with either monoclonal antibodies or polyclonal antisera. Immune cells that have undergone differentiation or activation can also be enumerated by staining for the presence of characteristic cell surface proteins by direct immunofluorescence in fixed smears of cultured cells.

Mature B cells can be measured in immunoassays, for example, by cell surface antigens including CD19 and CD20 with monoclonal antibodies labeled with fluorochromes or enzymes may be used to these antigens. B cells that have differentiated into plasma cells can be enumerated by staining for intracellular immunoglobulins by direct immunofluorescence in fixed smears of cultured cells.

Immunoglobulin Production Assay: B cell activation results in small, but detectable, quantities of polyclonal immunoglobulins. Following several days of culture, these immunoglobulins may be measured by radioimmunoassay or by enzyme-linked immunosorbent assay (ELISA) methods.

B cells that produce immunoglobulins can also be quantified by the reversed hemolytic plaque assay. In this assay, erythrocytes are coated with goat or rabbit anti-human immunoglobulins. These immunoglobulins are mixed with the activated immunoglobulin-producing lymphocytes and semisolid agar, and complement is added. The presence of hemolytic plaques indicates that there are immunoglobulin-producing cells.

Chemotactic Factor Assay: Chemotactic factors are molecules which induce or inhibit immune cell migration into or out of blood vessels, tissues or organs, including cell migration factors. The chemotactic factors of immune cells can be assayed by flow cytometry using labeled monoclonal antibodies to the chemotactic factor or factors being assayed. Chemotactic factors may also be assayed by ELISA or other immunoassays, bioassays, messenger RNA levels, and by direct measurements, such as cell counting, of immune cell movements in specialized migration chambers.

Addback Assays: When added to fresh peripheral blood mononuclear cells, autologous ex vivo activated cells exhibit an enhanced response to a “recall” antigen, which is an antigen to which the peripheral blood mononuclear cells had previously been exposed. Primed or stimulated immune cells should enhance other immune cells response to a “recall” antigen when cultured together. These assays are termed “helper” or “addback” assays. In this assay, primed or stimulated immune cells are added to untreated, usually autologous immune cells to determine the response of the untreated cells. The added primed cells may be irradiated to prevent their proliferation, simplifying the measurement of the activity of the untreated cells. These assays may be particularly useful in evaluating cells for blood exposed to virus. The addback assays can measure proliferation, cytokine production, and target cell lysis as described herein.

The above-described methods and other additional methods to determine an immune response are well known in the art.

### IV. Kits

The compositions can be packaged in a kit. Typically, the kit includes one or more of 1) a device for mechanically disrupting a subject’s epidermal tissue; 2) an intact, non-replicating or replication-impaired poxvirus; and 3) a T cell antigen or vector expressing the T cell antigen. The T cell antigen, or vector expressing the T cell antigen in an amount sufficient to stimulate an immune response when administered to disrupted epidermal tissue.

The compositions can be packaged in single or multi-vial kits that contain all of the components needed to prepare the complexes. A multi-vial kit preferably contains the same general components but employs more than one vial in reconstituting the compositions. In some embodiments, the contents of one or more vials are lyophilized and/or required to be frozen. Thus, in some embodiments, the contents of one or more vials are reconstituted and/or thawed prior to administration to a subject.

In one embodiment, a kit is provided comprising one or more containers filled with one or more of the following components: a live, non-replicating or replication-impaired virus, a T cell antigen or vector expressing the T cell antigen, and optionally comprising a co-stimulatory molecule, either in dried form (e.g. lyophilized), as a salt, or in a solution, optionally a solution or gel to dissolve or admix the components, and optionally an additional adjuvant. In some embodiments, the kits additionally contain a device for disrupting the epidermis. Associated with such a kit can be instructions on how to use the kit and optionally a notice in the form prescribed by a governmental agency regulating the manufacture, use or sale of pharmaceuticals or biological products, which notice reflects approval by the agency of manufacture, use or sale for human administration.

The kit also can contain stabilizers, bulking agents such as mannitol, that are designed to aid in the freeze-drying process, and other additives known to those skilled in the art.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method for increasing a T cell mediated immune response to a T cell antigen comprising
mechanically disrupting the epithelial tissue of a subject while administering (a) intact, non-replicating or replication-impaired poxvirus; and
(b) optionally, a T cell antigen, or a nucleic acid expressing the T cell antigen,
wherein the poxvirus does not express the T-cell antigen.

2. The method of claim 1, wherein the intact, non-replicating or replication-impaired poxvirus is derived by natural or artificial modification of a poxvirus.

3. The method of claim 2, wherein the poxvirus is selected from the group consisting of derivatives of orthopox, suipox, avipox, capripox, leporipox, parapoxvirus, molluscpoxvirus, and yatapoxvirus.

4. The method of claim 3, wherein the orthopox virus is a vaccinia virus.

5. The method of claim 4, wherein the vaccinia virus is selected from the group consisting of Modified Vaccinia Ankara (MVA), Wyeth strain, WR strain, NYCBH strain, ACAM2000, Lister strain, LC16m8, Elstree-BNm, Copenhagen strain, and Tiantan strain.

6. The method of claim 5, wherein the poxvirus is Modified Vaccinia Ankara (MVA).

7. The method of claim 1, wherein the T cell antigen is selected from the group consisting of a protein, a polypeptide, and nucleic acid encoding the antigen.

8. The method of claim 1, wherein a nucleic acid encoding the T cell antigen is co-administered with the poxvirus.

9. The method of claim 8, wherein the T cell antigen is a MHC class I peptide epitope.

10. The method of claim 1, wherein the T cell antigen is administered in the form of an agent selected from the group consisting of a plasmid, a cosmid, replicon RNA, a viral vector a virus-like-particle (VLP), liposomal nucleic acid, a prokaryotic cell, a fungal cell, an eukaryotic cell, and an artificial chromosome.

11. The method of claim 1, wherein T cell antigen is administered with the poxvirus.

12. The method of claim 1, wherein the T cell antigen is an endogenous antigen at or near the site of administration.

13. The method of claim 1 wherein the T cell antigen is a tumor-associated antigen (TAA), a tumor-specific antigen (TSA), or a tissue-specific antigen.

14. The method of claim 13, wherein the tumor-associated antigen is a tumor neoantigen.

15. The method of claim 13, wherein the tumor is in, or is derived from epithelial tissue selected from the skin, such as melanoma, oral mucosa, esophagus, reproductive mucosa and urogenital mucosa.

16. The method of claim 1, wherein the T cell antigen is derived from or raises an immune response against a cancer selected from the group consisting of melanoma, squamous cell carcinoma, basal cell carcinoma, Merkel cell carcinoma, adenexal carcinoma, cutaneous T or B cell lymphoma, sarcomas, adenocarcinoma, prostate adenocarcinoma, prostatic intraepithelial neoplasia, squamous cell lung carcinoma, lung adenocarcinoma, small cell lung carcinoma, ovarian cancer of epithelial origin, colorectal adenocarcinoma and leiomyosarcoma, stomach adenocarcinoma and leiomyosarcoma, hepatocellular carcinoma, cholangiocarcinoma, ductal adenocarcinomas of pancreas, endocrine pancreatic tumors, renal cell carcinoma, transitional cell carcinoma of kidney and bladder, and bladder squamous cell carcinoma.

17. The method of claim 1, wherein the subject has or is at risk of developing a viral, bacterial, fungal, or protozoal infection, and
wherein the T cell antigen is a viral, bacterial, fungal, or protozoal antigen.

18. The method of claim 17, wherein the T cell antigen is derived from, or raises a protective immune response against a pathogen selected from the group consisting of coronavirus, human immunodeficiency virus, influenza, dengue, Hepatitis A virus, Hepatitis B virus, Hepatitis C virus, human papilloma virus, Ebola, Marburg, Rabies, Hanta virus infection, West Nile virus, SARS-like Coronaviruses, Herpes simplex virus, Varicella-zoster virus, Epstein-Barr virus, Human herpesvirus, Alpha viruses, and St. Louis encephalitis, Mycobacterium tuberculosis, Salmonella typhi, Bacillus anthracis, Yersinia perstis, Francisella tularensis, Legionella, Chlamydia, Rickettsia typhi, and Treponema pallidum, Coccidioides immitis, Blastomyces dermatitidis, Cryptococcus neoformans, Candida albicans, and Aspergillus species, Plasmodium falciparum, Plasmodium vivax, Plasmodium ovale, Plasmodium malariae, Leishmania species, African Trypanosome species, American Trypanosome species, cryptosporidiums, isospora species, Naegleria fowleri, Acanthamoeba species, Balamuthia mandrillaris, Toxoplasma gondii, and Pneumocystis carinii.

19. The method of claim 18, wherein the T cell antigen is derived from, or raises a protective immune response against a SARS-Cov-2 virus related b coronaviruses.

20. The method of claim 1, wherein the epithelial skin tissue is disrupted without penetrating the entire epidermis, and wherein the non-replicating or replication-impaired poxvirus and the T cell antigen, or vector expressing the T cell antigen are administered to the stratum corneum.

21. The method of claim 1, wherein the epithelial tissue is mechanically disrupted by one or more selected from the group consisting of a scarification needle, a hypodermic needle, an abrader and microneedles.

22. The method of claim 1, wherein the epithelial tissue is disrupted essentially at the same time as the administration of the poxvirus and the T cell antigen, or a vector expressing the T cell antigen.

23. The method of claim 1, wherein the epithelial tissue is disrupted before administration of the poxvirus and the T cell antigen, or the vector expressing the T cell antigen.

24. The method of claim 1, wherein the poxvirus is delivered to a first disrupted epithelial tissue location on the subject and the T cell antigen, or vector expressing the T cell antigen is delivered to a second disrupted epithelial tissue location of the subject.

25. The method of claim 24, wherein the type of epithelial tissue of the first disrupted epithelial tissue location is different from that of the second disrupted epithelial tissue location.

26. The method of claim 1, further comprising administering to the subject, or co-expressing within the subject a molecule selected from the group consisting of a co-stimulatory molecule, a growth factor, and a cytokine.

27. The method of claim 26, wherein the molecule is selected from the group consisting of:
IL-1a or b, IL-2, IL-7, IL-12, IL-15, IL-18, IL-23, IL-27, B7-2, B7-H3, CD40, CD40L, ICOS-ligand, OX-40L, 4-1BBL, GM-CSF, SCF, FGF, Fantigen-ligand, CCR4.

28. The method of claim 26, wherein the molecule is administered to the subject at the same time or after the poxvirus and T cell antigen is administered.

29. The method of claim 1, wherein the molecule is administered to the subject to the same site that the T cell antigen is administered or at a distant site.

30. A kit comprising
an intact, non-replicating or replication-impaired poxvirus, and
a T cell antigen, or vector expressing the T cell antigen,
wherein the poxvirus is in an amount sufficient to stimulate an immune response when administered to disrupted epidermal tissue.

31. The kit of claim 30, wherein the kit further comprises a device for mechanically disrupting a subject’s epidermal tissue.

32. A dosage unit for inducing or stimulating a protective T cell mediated immune response to a T cell antigen comprising an intact, non-replicating or replication-impaired poxvirus encoding the T cell antigen in an effective amount to increase the immune response to the antigen when administered with mechanical disruption of an epidermal tissue.
